(19) United States
(12) Patent Application Publication
(10) Pub. No.: US 2023/0405177 A1
(43) Pub. Date: Dec. 21, 2023

(54) ULTRASHORT TETRAMERIC PEPTIDE NANOGELS SUPPORT TISSUE GRAFT FORMATION, WOUND HEALING AND 3D BIOPRINTING

(71) Applicant: KING ABDULLAH UNIVERSITY OF SCIENCE AND TECHNOLOGY, Thuwal (SA)

(72) Inventors: Charlotte A.E. HAUSER, Thuwal (SA); Hepi Hari SUSAPTO, Thuwal (SA)

(21) Appl. No.: 18/218,192

(22) Filed: Jul. 5, 2023

Related U.S. Application Data
(63) Continuation-in-part of application No. 17/401,542, filed on Aug. 13, 2021.
(60) Provisional application No. 63/067,962, filed on Aug. 20, 2020.
(60) Provisional application No. 63/067,913, filed on Aug. 20, 2020.
(60) Provisional application No. 63/358,563, filed on Jul. 6, 2022.
(60) Provisional application No. 63/523,658, filed on Jun. 28, 2023.

(51) Int. Cl. A61L 15/32; C07K 9/00

(52) U.S. Cl. CPC A61L 15/32; C07K 9/001

(57) ABSTRACT

Newly developed peptide nanogels provide native cues to human dermal fibroblasts as well as mouse myoblast cells and promote proliferation and extensive network formation in vitro is presented. The results represent an improvement in the fabrication of dermal grafts as well as 3D skin models. In addition, the application of these ultrashort peptide nanogels on full-thickness wounds in a minipig model demonstrated biocompatibility with the minipig skin tissue, as the peptide nanogels did not trigger wound inflammation. Thus, they can be considered as a safe biomaterial for topical applications. It is shown that both peptides are printable. The ability to print peptides and the return of high cell viability within the printed construct will open up the possibility of 3D bioprinting of different cell types in future.

### CROSS-REFERENCE TO RELATED APPLICATIONS

This application claims benefit of priority of U.S. patent application Ser. No. 17/401,542 entitled, “SCAFFOLDS FROM SELF-ASSEMBLING TETRAPEPTIDES SUPPORT 3D SPREADING, OSTEOGENIC DIFFERENTIATION AND ANGIOGENESIS OF MESENCHYMAL STEM CELLS” filed Aug. 13, 2021, which in turn claims priority to U.S. Provisional Patent Application No. 63/067,913, entitled “PEPTIDE COMPOUND WITH REPETITIVE SEQUENCE” filed Aug. 20, 2020 and to U.S. Provisional Patent Application No. 63/067,962, entitled “TETRAMERIC SELF-ASSEMBLING PEPTIDES SUPPORT 3D SPREADING AND OSTEOGENIC DIFFERENTIATION OF MESENCHYMAL STEM CELLS” filed Aug. 20, 2020, of which the present application is a continuation-in-part application. This application also claims priority to U.S. Provisional Patent Application No. 63/358,563, entitled “SELF-ASSEMBLING PEPTIDES FOR DRUG DELIVERY APPLICATIONS” filed Jul. 6, 2022, and to U.S. Provisional Patent Application No. 63/523,658, entitled “DESIGN AND DEVELOPMENT OF PEPTIDE-MODIFIED ANTINEOPLASTIC DRUGS FOR TARGETING BREAST CANCER CELLS” filed Jun. 28, 2023. The entire contents and disclosures of these patent applications are incorporated herein by reference in their entirety.

This application refers to “ULTRASHORT TETRAMERIC PEPTIDE NANOGELS SUPPORT TISSUE GRAFT FORMATION, WOUND HEALING AND 3D BIOPRINTING,” in Peptide-based Biomaterials journal published on Nov. 18, 2020. The entire contents and disclosures of these patent applications are incorporated herein by reference.

### BACKGROUND

### Field of the Invention

The present invention relates to generally to peptide nanogels supporting tissue graft formation, wound healing and 3D bioprinting.

### Background of the Invention

Every year, all over the world, hundreds of thousands of patients require hospitalization as a result of organ failure, which affects quality of life and is costly to treat. Tissue engineering is an alternative approach for creating tissue constructs and fabricating functional organs using biological scaffolds. There is still a need to find more appropriate materials that can substitute for compromised skin and muscle tissue and that demonstrate superior applicability compared to autografts.

### SUMMARY

According to first broad aspect, the present invention provides an ultrashort self-assembling amphiphilic peptide wherein said aliphatic amino acids and said aromatic amino acids and said polar amino acids are either D amino acids or L amino acids, comprising at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

According to a second broad aspect, the present invention provides a method of wound treatment comprising applying a material to a subject, wherein the material comprises an ultrashort self-assembling amphiphilic peptide wherein said aliphatic amino acids and said aromatic amino acids and said polar amino acids are either D amino acids or L amino acids, comprising at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

According to a third broad aspect, the present invention provides a method of preparing a hydrogel or organogel, the method comprising dissolving a peptide in an aqueous solution or an organic solution, respectively, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

According to a fourth broad aspect, the present invention provides a kit of parts, the kit comprising a first container with a peptide and a second container with an aqueous or organic solution, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

According to a fifth broad aspect, the present invention provides a kit comprising an effective amount of a material, wherein the material comprises an ultrashort self-assembling amphiphilic peptide wherein said aliphatic amino acids and said aromatic amino acids and said polar amino acids are either D amino acids or L amino acids, comprising at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group, and wherein the material is applied in at least one selected from the group consisting of 2D-3D printing and 2D-3D molding.

According to a sixth broad aspect, the present invention provides a wound dressing or wound healing agent comprising a hydrogel or organogel comprising a peptide, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

According to a seventh broad aspect, the present invention provides a pharmaceutical composition comprising an ultrashort self-assembling amphiphilic peptide wherein said aliphatic amino acids and said aromatic amino acids and said polar amino acids are either D amino acids or L amino acids, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

According to an eighth broad aspect, the present invention provides a method of wound treatment comprising administering a hydrogel or organogel comprising a peptide to a subject via a device, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The accompanying drawings, which are incorporated herein and constitute part of this specification, illustrate exemplary embodiments of the invention, and, together with the general description given above and the detailed description given below, serve to explain the features of the invention.

FIG. 1 illustrates molecular dynamics simulations showing the process of self-assembly of ultrashort amphiphilic peptide monomers to dimer structures, and further to fibrils, which then assemble further to supramolecular networks of condensed fibers according to one embodiment of the present invention.

FIG. 2 illustrates ultrashort peptides self-assembled into three-dimensional nanofibrous networks according to one embodiment of the present invention.

FIG. 3 illustrates SEM images of fibroblasts grown on 3D scaffolds and proliferation and adhesion of HDFn within the scaffolds according to one embodiment of the present invention.

FIG. 4 illustrates overlaid confocal fluorescent images of fibroblast cells encapsulated in peptide hydrogels and microscopic images for cryosection of 3D-cultured HDFn in IVFK and IVZK nanogels according to one embodiment of the present invention.

FIG. 5 illustrates the vascularization of co-cultured human skin fibroblasts and HUVECs within different scaffolds according to one embodiment of the present invention.

FIG. 6 illustrates a dropper/closure device for administering a hydrogel or organogel comprising the peptide according to one embodiment of the present invention according to one embodiment of the present invention.

FIG. 7 illustrates a squeeze bottle pump spray device for administering a hydrogel or organogel comprising the peptide according to one embodiment of the present invention according to one embodiment of the present invention.

FIG. 8 illustrates an airless and preservative-free spray device for administering a hydrogel or organogel comprising the peptide according to one embodiment of the present invention according to one embodiment of the present invention.

FIG. 9 illustrates an injectable device for administering a hydrogel or organogel comprising the peptide according to one embodiment of the present invention according to one embodiment of the present invention.

FIG. 10 illustrates a patch device for administering a hydrogel or organogel comprising the peptide according to one embodiment of the present invention according to one embodiment of the present invention.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and information on the internet can come and go, but equivalent information can be found by searching the internet.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present invention, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present invention, the term “comprising”, the term “having”, the term “including,” and variations of these words are intended to be open-ended and mean that there may be additional elements other than the listed elements.

For purposes of the present invention, directional terms such as “top,” “bottom,” “upper,” “lower,” “above,” “below,” “left,” “right,” “horizontal,” “vertical,” “up,” “down,” etc., are used merely for convenience in describing the various embodiments of the present invention. The embodiments of the present invention may be oriented in various ways. For example, the diagrams, apparatuses, etc., shown in the drawing figures may be flipped over, rotated by 90° in any direction, reversed, etc.

For purposes of the present invention, a value or property is “based” on a particular value, property, the satisfaction of a condition, or other factor, if that value is derived by performing a mathematical calculation or logical decision using that value, property or other factor.

For purposes of the present invention, the term “bioinks” as used herein means materials used to produce engineered/artificial live tissue using 3D printing.

For purposes of the present invention, the term “effective amount” or “effective dose” or grammatical variations thereof refers to an amount of an agent sufficient to produce one or more desired effects. The effective amount may be determined by a person skilled in the art using the guidance provided herein.

For purposes of the present invention, the term “gel” and “hydrogel” are used interchangeably. These terms refer to a network of polymer chains, entrapping water or other aqueous solutions, such as physiological buffers, of over 99% by weight.

For the purposes of the present invention, the term “patch” refers to adhesive patch that is placed on the skin or surfaces.

For purposes of the present invention, the term “scaffolds” as used herein means the ultra-short peptide or other polymer materials in the bioinks that provide support for the cellular components.

For purposes of the present invention, the term “subject” and the term “patient” refers to an entity which is the object of treatment, observation, or experiment. By way of example only, a “subject” or “patient” may be, but is not limited to a human, a mammal, a reptile, a bird, a fish, an amphibian, and an invertebrate.

For purposes of the present invention, the term “therapeutically effective amount” and the term “treatment-effective amount” refers to the amount of a drug, compound or composition that, when administered to a subject for treating a disease or disorder, or at least one of the clinical symptoms of a disease or disorder, is sufficient to affect such treatment of the disease, disorder, or symptom. A “therapeutically effective amount” may vary depending, for example, on the compound, the disease, disorder, and/or symptoms of the disease or disorder, severity of the disease, disorder, and/or symptoms of the disease or disorder, the age, weight, and/or health of the subject to be treated, and the judgment of the prescribing physician. An appropriate amount in any given instance may be readily ascertained by those skilled in the art or capable of determination by routine experimentation.

For purposes of the present invention, the term “ultra-short peptide” and “self-assembling peptide” are used interchangeably. These terms refer to a sequence containing 3-7 amino acids.

### DESCRIPTION

While the invention is susceptible to various modifications and alternative forms, specific embodiment thereof has been shown by way of example in the drawings and will be described in detail below. It should be understood, however that it is not intended to limit the invention to the particular forms disclosed, but on the contrary, the invention is to cover all modifications, equivalents, and alternatives falling within the spirit and the scope of the invention.

### The Potential of Tissue Engineering

There are many ailments that plague human beings. Among them are the diseases that destroy tissue and lead to organ failure, such as cancer and diabetes.¹ These illnesses affect the quality of life and are costly to treat and may eventually cause fatalities. From a historical prospect, the conventional treatment for loss of organs (tissues) is organ transplantation, in which the damaged organ is replaced by a healthy, functional organ. However, this treatment is not sufficient to serve all patients based on need due to a shortage of healthy donor tissue. Thus, there are a huge number of patients on waiting lists for organ transplantation. The “gold standard” and only suitable option for the treatment of skin or muscle damage is a method known as a grafting.²In the case of an autograft, a piece of healthy tissue, directly derived from the patient, is utilized, which contains the whole outer layer and a minor portion of the middle layer³ of the tissue. Autograft remains the most frequently used grafting material as the body generally recognizes the graft as its own tissue and does not elicit any adverse immunological reaction. However, the utilization of grafts is limited in cases of full-thickness skin wounds that are associated with a complete loss of the epidermis and dermis as well as volumetric muscle loss, leading to insufficient autografts to cover the whole area. Furthermore, there may be a lack of healthy donor sites or limits on their availability.⁴ In addition, autografts can cause pain, bleeding, infection, nerve damage, scar formation, and, in some cases, a repeat of the grafting procedure may still be required. Alternatively, allografts, i.e tissue grafts from another person, or xenografts, i.e tissue grafts derived from animals, have been used for wound coverage, but these grafts are only suitable as a temporary wound covering, as they are associated with disease transmission and immune rejection.⁵

In 1993, Langer and Vacanti coined the term “tissue engineering” for a procedure that combines the principles of cell culture, molecular biology, and chemical engineering in in vitro approaches to address the aforementioned tissue shortage issue.⁶ This technology is a promising method that allows for the reproduction of natural structures that mimic native tissue.

In the past, in vitro tissue models have been developed without the need for healthy donor tissue using cell monomer structures by two-dimensional (2D) cell culturing.⁷ However, these models do not accurately copy or mimic the natural tissue, as the cells in the body grow in a three-dimensional (3D) environment. Cells embedded within the extracellular matrix in a 2D or a 3D environment show significant differences in cellular functions. Thus, 2D and 3D environments affect cellular cues as well as adhesion, proliferation, and differentiation properties.⁸⁻¹⁰ Therefore, researchers have fabricated more advanced in vitro tissue models that more closely resemble the natural structures found in the body, providing more reliable results than the 2D models previously developed.¹⁰ These novel tissue models were developed to create alternative tissue constructs and fabricate functional organs relying on known biomaterials. They were usually denoted as scaffolds, matrices or constructs that can integrate into the host tissue. The scaffold is a major component of a tissue-engineered construct, which is made from a biomaterial with a 3D architecture, to accommodate more cells, promote cell adhesion and proliferation. Recently, fabrication of various biomimetic scaffolds has attracted the interest of a large number of researchers.^{11, 12}

Biomaterials used in tissue engineering can be either natural biomaterial-based hydrogels, originated from organic compounds produced in nature, or synthetic biomaterial-based hydrogels, derived from artificial materials, such as polymers, or synthesized from natural amino acids found in bodies, such as peptides. The design of novel synthetic biomaterials aims to remodel the structures of the native architecture and the functionality of the extracellular matrix.¹³

### Natural Biomaterial-Based Hydrogels

There are many natural biomaterials, and these were the first to be used in tissue engineering. The most popular materials among these are collagen, gelatin, chitosan, fibrin, alginate and hyaluronic acid. While natural biomaterial-based hydrogels are similar to the natural extracellular matrix (ECM), are biocompatible and are capable of supporting cellular adhesion, there are several drawbacks to their use. The disadvantages of these natural biomaterials include excessive contraction, poor vascularization, scar formation and, most importantly, poor mechanical properties, rapid degradation and batch-to-batch variation.¹⁴⁻¹⁶

### Synthetic Biomaterial-Based Hydrogels

### Synthetic Polymer-Based Hydrogels

Due to the poor mechanical properties and rapid degradation rates associated with natural biomaterials, scientists have turned their attention to synthetic polymer-based hydrogels as they allow for the manipulation of these properties. Many mainly polymeric porous biomaterial scaffolds have been fabricated and used in numerous tissue engineering applications.¹⁷⁻²³ However, these products are more likely to produce an immune response in the form of inflammation,²⁴ and the associated acid degradation by products can negatively affect or harm the cellular environment and can lead to delays in the tissue regeneration process. The synthetic biomaterials used most frequently are polyethylene glycol, polyvinyl alcohol, polycaprolactone and polylactic acid.

### Synthetic Peptide-Based Hydrogels

Given the limitations of current synthetic polymers, the fabrication of rationally designed peptide hydrogels or biomimetic scaffolds has attracted considerable attention due to their synthetic, but natural, background.” These biomaterials possess some unique advantages over synthetic polymers, such as true nanofibrous structures, which is a major requirement for tissue regeneration,^{25, 26} and thus they better mimic native ECM components with respect to both architecture and mechanical properties.²⁷

Biomaterial-based hydrogels made from self-assembling ultrashort peptides are promising biomaterials for a variety of biomedical applications, such as tissue engineering, drug delivery, regenerative medicine, microbiology and biosensing.²⁸⁻³¹ Among these biomaterials are nanofibrous scaffolds, which allow the engineered cellular structures to merge with existing ECM fibers by providing additional structural and functional support with minimal rejection.¹³ It has been reported that the nano-topographical features of the scaffold affect the cellular behavior. Therefore, modulating nanostructured scaffolds towards a more suitable surface topography and more desirable chemical functionalities may overcome the limitations associated with natural and synthetic polymeric biomaterials.^{32, 33} Many hydrogels have been used and evaluated for their cellular activity, mechanical properties and myogenic potential.³⁴⁻³⁷ However, there is still a need to find more appropriate materials that are able to substitute for compromised skin and muscle tissue and that demonstrate superior applicability compared to autografts.

### Design Criteria for Smart Ultrashort Self-Assembling Amphiphilic Peptides and Mechanism of Self-Assembly

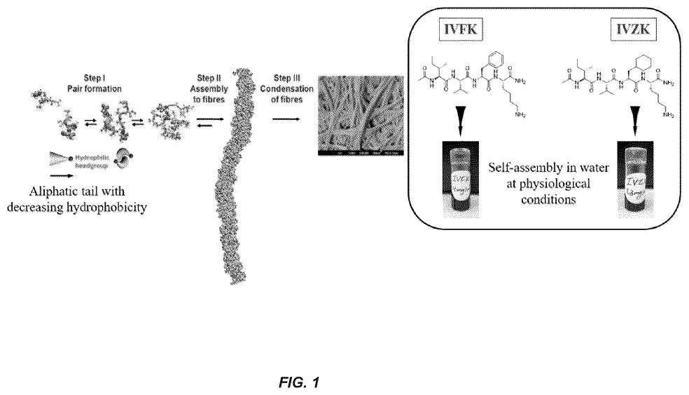

In an effort to overcome these limitations, the Hauser laboratory has rationally designed and fabricated ultrashort self-assembling amphiphilic peptides with bio-mimetic properties that demonstrate biocompatibility, non-cytotoxicity and non-immunogenicity. Novel peptide compounds, containing four natural amino acids, possess a comparative cost advantage over many reported protein or peptide-based hydrogels due to their short length. These so-called ultrashort peptide compounds are synthesized from natural amino acids that possess a unique motif found in the body. The peptide compounds can form nanofibrous structures that mimic those of native collagen by the process of self-assembly. These peptides have an innate tendency to self-assemble into helical nanofibers under aqueous conditions without the need for gelators, cross-linkers or mechanical stimulators. The helical nanofibers organize themselves into supramolecular 3D-meshed nanofibrous networks, with an average fiber diameter of around 10-20 nm, and where the fibers structurally resemble collagen fibers with respect to topography.²⁷ The self-assembly of these tetrameric peptides is assumed to occur through antiparallel pairing interactions of two peptide monomers, supported by the formation of helical intermediate structures, as had been demonstrated earlier.²⁸ Condensation of these fibers leads to hydrogel scaffold formation (FIG. 1).

Ultrashort self-assembling amphiphilic peptides have been used as biomaterials in regenerative medicine applications and as matrices for the delivery of encapsulated bioactive molecules for therapeutic applications.^{28, 38-41} The peptide scaffolds have been applied in skin tissue regeneration to achieve a significant rate of wound closure and repair without the addition of any growth factors.^{42, 43}

Disclosed embodiments primarily focus on biomedical applications using two novel peptide hydrogels assembled from tetrameric amphiphilic ultra-short peptide compounds:^{31, 41} Ac-IVFK-NH₂ (Ac-Ile-Val-Phe-Lys-NH₂) and Ac-IVZK-NH₂ (Ac-Ile-Val-Cha-Lys-NH₂). Disclosed embodiments aim to use these peptide hydrogels for the purpose of skin tissue engineering and muscle tissue engineering, combining the advantages of using hydrogels that are both natural and synthetic. When rationally designing this biomaterial, the guiding criterion was to generate amphiphilic peptide sequences with a hydrophobic tail and a hydrophilic head group. The characteristic motif of the peptide sequences holds the cues required for non-covalent interactions that drive molecular recognition and self-assembly. Also, disclosed embodiments have replaced the aromatic phenylalanine residue in IVFK with its aliphatic amino acid counterpart, cyclohexyl alanine, to give IVZK, in order to explore the effect of sequence modification on self-assembly and supramolecular organization when exchanging an aromatic residue with a non-aromatic residue.

### Peptides Synthesis and Vial Inversion Assays (Hydrogel Formation)

The synthesis of both peptides, Ac—IVFK—NH₂ (Ac-Ile-Val-Phe-Lys-NH₂) and Ac-IVZK-NH₂ (Ac-Ile-Val-Cha-Lys-NH₂), by Fmoc-based solid-phase peptide synthesis on rink amide resin is an easy, fast and cost-effective method due to the small size of the peptide compounds. The gelation of both peptides occurred within a few minutes at a minimum concentration of 4 mg mL⁻¹ and 3 mg mL⁻¹ for IVFK and IVZK, respectively, as shown in FIG. 1. The final volume ratio of peptide solution to 10×phosphate-buffered saline solution was 9:1.

Molecular dynamics simulations show the process of self-assembly of ultrashort amphiphilic peptide monomers to dimer structures, and further to fibrils, which then assemble further to supramolecular networks of condensed fibers as seen in the field emission scanning electron microscope image (right picture from FIG. 1). Ultrashort peptides self-assemble into nanofibers that form three-dimensional nanogels.²⁹ Reproduced from Ref. 29 with permission from PNAS, Copyright 2010. The self-assembling peptides IVFK (4 mg mL⁻¹) and IVZK (3 mg mL⁻¹) generate macromolecular nanofibrous hydrogels in an aqueous solution.¹¹² Reproduced from Ref. 112 with permission from Whioce Publishing PTE Ltd, Copyright 2019 (FIG. 1).

### Surface Topography and Morphology of Peptide Hydogels

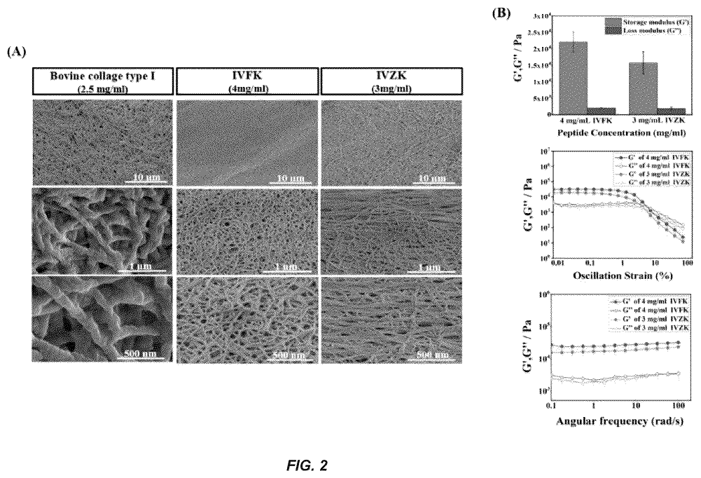

FIG. 2 illustrates ultrashort peptides self-assemble into three-dimensional nanofibrous. FIG. 2(A) illustrates field emission scanning electron microscopy images of 2.5 mg mL⁻¹ bovine collagen type I, 4 mg mL⁻¹ IVFK and 3 mg mL⁻¹ IVZK nanogels, at different magnifications. The nanofibrous morphology of the self-assembling peptides was demonstrated using scanning electron microscopy (SEM) and the images were compared to those observed in bovine collagen (FIG. 2, (A) left), which is comprised of a unique triple-helical structure.⁴⁴ The detailed assessment of IVFK nanogels (FIG. 2, (A) middle) and IVZK nanogels (FIG. 2, (A) right) show that the fibrous structures of the peptide networks resemble the fibrous structure of collagen. The assembly of the peptide compounds to supramolecular structures in the form of nanofibrous scaffolds is supported by the antiparallel pairing of two peptide monomers (see FIG. 1). Subsequently, elongated fibers form because of further assembly of the antiparallel-arranged peptide pairs, through stacking of those pairs. Condensation of fibrils to fibers resulted in hydrogel formation. The diameter of the nanoscale fibers are within the range found in the natural ECM (5-300 nm).⁴⁵ A distinctive feature of the nanofibrous structure allows the scaffold to entrap >99% of water within its bulk volume, forming a soft solid matter or soft solid hydrogel; thus, the hydrogel preserves a moist environment. When applied to burn wounds in an animal model, the wound tissue remained hydrated, which in turn reduced pain during the frequent dressing changes.⁴² Disclosed results support the use of ultrashort peptide hydrogels as scaffolds for skin regeneration,⁴⁶⁻⁴⁹ particularly for full-thickness wounds.

### Mechanical Properties and Rheology Measurements

FIG. 2 (B) illustrates rheology result of 4 mg mL⁻¹ IVFK and 3 mg mL⁻¹ IVZK peptide nanogels. Equilibrium moduli at 5 minutes, oscillatory frequency sweep test and oscillatory amplitude sweep test are shown, respectively.¹¹¹ Reproduced from Ref. 111 with permission from Whioce Publishing PTE Ltd, Copyright 2018. The viscoelastic properties of peptide nanogels were characterized by using oscillatory rheology measuring the storage modulus (G′) and loss modulus (G″). Samples were examined through time-sweep analysis in a linear visco-elastic range (LVR) for 5 minutes by keeping the storage modulus constant under elastic deformation. The G′ values of IVFK and IVZK nanogels were found to be approximately 22.1 kPa and 15.8 kPa, respectively, while their G″ values were less than their G′ values, indicating the gel state of both samples, as shown in FIG. 2 (B).⁵⁰ Both samples also showed frequency-independent behavior, without any crossover at lower frequencies, which is a common viscoelastic property for hydrogels (FIG. 2 (B)).⁵¹ An amplitude sweep test demonstrated the length of the LVR under increasing strain values. It was found that the LVR for both peptide gels was still constant under 0.2% strain. However, the later crossover point of IVFK, where G′=G″, indicates that IVFK can tolerate higher strain before breaking, in addition to its higher G′ value (FIG. 2 (B)).⁵²

### Biomedical Applications of Self-Assembling Hydrogels

### Peptide Nanogels as Scaffold for Fabricating 3D Dermal Grafts for Skin Tissue Engineering

The outer covering and the largest organ of the body is the skin, which can control several physiological functions, including temperature regulation, dehydration prevention and internal fluid loss, via the opening and closing of pores and perspiration. Furthermore, it acts as a barrier and plays an essential role in protecting the body against pathogens and mechanical impact. However, skin can be lost due to infection, trauma or disease.⁵³ The skin's natural healing capacity is usually sufficient for it to repair itself following damage as long as the affected area measures less than 4 cm in diameter and the person is not subject to underlying genetic abnormalities or diseases that affect wound healing.⁵³ However, in severe skin injuries or full-thickness wounds that affect the dermis, and possibly the hypodermis, the epithelial cells are unable to migrate and tissue contraction cannot cover the wound. To protect the injured layers of skin and allow restoration of the damaged portion, the edges of the damaged surface require suturing together or surgical skin replacement may be required. Effective wound healing is essential to maintain homeostasis and prevent the penetration of infectious agents.^{54, 55} Detailed molecular, biological and physiological assessments of the specific wound-healing effects provided by the standard-of-care wound dressing materials are mostly missing. As such, replacements for diseased and damaged skin tissues are in high demand.^{6, 56, 57} Therefore, disclosed embodiments have used the aforementioned hydrogels, which are made from self-assembling ultrashort peptides with promising properties for wound healing for the following purposes: to characterize the disclosed newly designed nanogels and to test their effect on skin cell proliferation. Further, disclosed embodiments may test the ability of these materials for use as scaffolds, particularly for the fabrication of 3D skin grafts needed for wound healing. Finally, disclosed embodiments may evaluate the efficacy of these materials with and without silver nanoparticles as dressings on minipigs with full-thickness excision wounds, compared to standard-of-care hydrogels.

### Biocompatibility of Self-Assembly Ultrashort Peptide Hydrogels on Skin Cells

Different biocompatibility assays were applied to test the effect of tetrameric self-assembly peptide nanogels on skin cell proliferation.

The metabolic activity of viable human dermal fibroblast cells (HDFn) in the presence of peptide hydrogels showed that there was no significant difference in cell viability with various peptide concentrations within 48 hours, compared to both positive and negative controls. However, a slight reduction in cell viability was detected with no cytotoxicity caused by either pep-tide hydrogels on both dermal and epidermal layers compared to the control Matrigel® (data not shown).

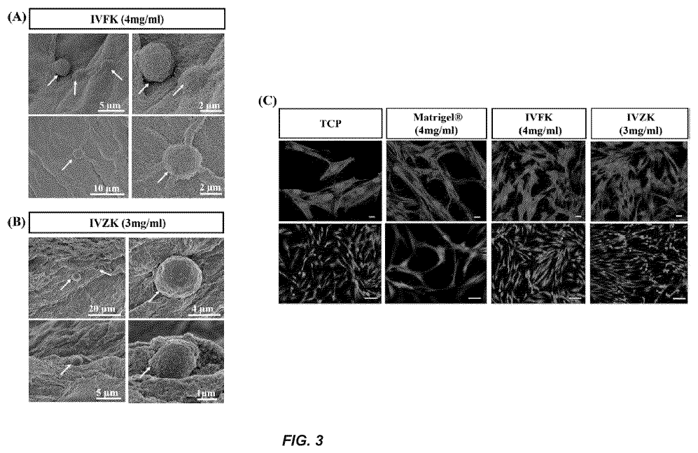

As cell adhesion and spreading over the scaffold are essential steps for wound healing and restoration of tissues, the cellular attachment and morphological changes of cells growing on 3D scaffolds were tested in a 3D micro-environment. FIG. 3 illustrates SEM images of fibroblasts grown on 3D scaffolds after 48-hour culture using 4 mg mL⁻¹ IVFK (A) and 3 mg mL⁻¹ IVZK (B). White arrows indicate the fibroblast cells. Proliferation and adhesion of HDFn within the scaffolds (4 mg mL⁻¹ IVFK and 3 mg mL⁻¹ IVZK nanogels) and cells cultured for 72 hours (C) are shown. Actin cytoskeleton fluorescence (top row) and cell tracker green fluorescence (bottom row) represent the morphology and the attachment of cells, respectively. Nucleus is shown in blue, and F-actin is shown in red. TCP: negative control tissue culture plate. Scale bars correspond to 50 μm (top row) and 20 μm (bottom row), respectively. The peptide hydrogels support the formation of extensive 3D fibroblast networks within 48 hours. SEM results showed that the cells were anchored on the surface of IVFK nanogels, while in IVZK nanogels the cells were located on the outer surface of the scaffold and not wholly speared into the nanofibers (FIG. 3 (A) and FIG. 3 (B)). As the SEM results did not adequately depict the adhesion and morphology of the cells cultured on the 3D scaffold, fluorescence microscopy was used to confirm cellular interaction and spreading over the scaffold. Phalloidin test results revealed a standard morphology of cells after 3 days of incubation with both nanofibrous scaffolds compared to Matrigel® and TCP (FIG. 3 (C)). Furthermore, cell tracker florescent staining verified the attachment and spreading of HDFn on both scaffolds compared to Matrigel® and TCP, indicating the superiority of these scaffolds (FIG. 3 (C)).

In addition, the mitochondrial activity of HDFn cells within 3D culture hydrogels confirmed that the proliferation of cells in 3D culture gradually increased with time. This test was accomplished by measuring the amount of ATP produced from viable cells, which reflects the number of metabolically active cells. It is worth mentioning that the majority of cells were metabolically active at 21 days and the rate of proliferation was comparable to the positive-control Matrigel® (data not shown). A small increase in cell number was also observed on days 14 and 21, this observation is reasonable and can be attributed to a high cell density, which may obstruct cellular growth.⁵⁸

Moreover, the cellular compatibility of peptides on cell-embedded hydrogel for 3, 7, 14 and 21 days revealed that most of the cells remained viable in both hydrogels throughout the 21 days, indicating that diffusion of nutrients and removal of waste products were sufficient to maintain cell viability. However, there were very few dead cells within the matrix. It is worth mentioning that this test verified that the reduction in cell viability is not due to the toxicity of the hydrogels and cell death, but to a change in the local cellular microenvironment and diffusion barrier.⁵⁹

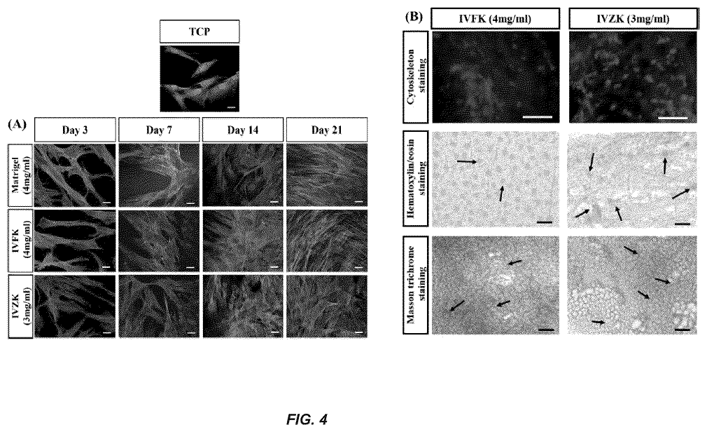

Furthermore, the morphology and growth conditions of the cells embedded in the hydrogels were investigated using staining with phalloidin. FIG. 4 (A) illustrates overlaid confocal fluorescent images of fibroblast cells encapsulated in peptide hydrogels (4 mg mL⁻¹ IVFK and 3 mg mL⁻¹ IVZK nanogels). Matrigel® (4 mg mL⁻¹) was used as positive control and cultured for different time points at days 3, 7, 14 and 21, respectively. Nucleus shown in blue, F-actin shown in red and vinculin in green. TCP: negative control tissue culture plate. Scale bars correspond to 20 μm.

Immunofluorescent staining of F-actin revealed a comparable stretched and extended F-actin on day 3 and day 7 in all tested conditions (FIG. 4 (A)). It is worth mentioning that after 21 days of culturing, prominent well organized, stretched and dense actin fibers were observed in IVFK nanogels and Matrigel®, while the fibers in IVZK nanogels were dense but not as well organized (FIG. 4 (A)). In addition, the number of cells in IVFK nanogels and Matrigel® was relatively higher when compared to IVZK nanogels (manuscript in preparation).⁶⁰ This could be attributed to the fact that IVFK provides native cues and offers more surface area for the cells to divide and grow. Also, this hydrogel may be sufficiently porous to accommodate more cells and to help in spreading and viability.

Additionally, histology studies confirmed the cellular distribution and network formation throughout the entire depth of the 3D-cultured HDFn construct.

FIG. 4 (B) illustrates microscopic images for cryosection of 3D-cultured HDFn in IVFK (4 mg mL⁻¹) and IVZK (3 mg mL⁻¹) nanogels. Actin/DAPI staining shows the cellular distribution and network formation. Black arrows show the nucleus distribution with H&E staining, and collagen I deposition in blue, after six days in culture. Immunohistochemistry results of a cryosection construct illustrated that the fibroblasts tended to spread and attach to each other in three dimensions (FIG. 4 (B)). This observation was further confirmed by hematoxylin and eosin (H&E) staining, which is commonly used in histology to provide basic structural visualization of tissues.61 H&E staining indicated random and uniform distribution of HDFn across the entire depth of the construct. In addition, Masson's trichrome staining for the density of collagen deposition within the 3D cultured construct revealed that HDFn cells start replacing the hydrogel with their cellular matrix, as shown in FIG. 4 (B), which might be the result of the efficient biodegradability of these hydrogels in vivo, through enzymatic hydrolysis. Overall, it was confirmed that both peptide hydrogels support cell viability, distribution, and network formation; demonstrating a suitable 3D microenvironment for skin tissue regeneration.

### Peptide Nanogels as Scaffold for Fabricating Artificial Skin Substitutes

After confirming the biocompatibility of these scaffolds on 3D-cultured HDFn cells, the major constituents of the middle dermal layer of the skin, artificial skin was constructed by co-culturing human epidermal keratinocytes (HEKn) with HDFn. Bright-field microscope observation of the direct contact of keratinocytes and fibroblasts revealed enhancement in keratinocyte proliferation (manuscript in preparation).⁶⁰

In addition, disclosed embodiments tracked and clearly distinguished the proliferation of co-cultured HDFn with HEKn by applying a different tracker fluorescent marker to the 3D co-cultured system. Homogeneous distribution of fibroblasts and keratinocytes in the peptide hydrogels was detected through confocal microscopy. A biomarker, cytokeratin 14 (CK14), was significantly expressed in keratinocytes that were highly proliferated and covered most of the fibro-blasts and tended to form skin-like structures on top of the fibroblasts (data not shown).

Furthermore, volume-scanning electron microscopy results confirmed artificial skin formation, including an epidermal layer. Also, formation of a spongy dermis layer was observed, with fibroblasts distributed across the entire section. These observations are consistent with the disclosed findings for cryosections of the middle dermis construct (FIG. 4 (B)).

Furthermore, the functionality of the artificial skin construct was con-firmed by measuring levels of different cytokines (IL-1, IL-6) and growth factors (TGB1 and β-FGF). This points to interactions between keratinocytes and fibroblasts. The underlying paracrine growth factor signaling is essential to maintain cutaneous homeostasis and skin regeneration. The quantitative expression of cytokines and growth factors revealed significantly higher levels of IL-1α, IL-6, TGF-β1 and β-FGF in the 3D-co-cultured system compared to the monoculture. However, little reduction in IL-1α and β-FGF was observed in the 2D-cultured system (TCP). In addition, IL-6, TGF-β1 and β-FGF were significantly expressed in 3D fibroblast monocultures and co-cultures, while IL-1α was significantly expressed in 3D keratinocyte monocultures and co-cultures (manuscript in preparation).⁶⁰

### Peptide Nanogels as Scaffold for Fabricating 3D Vascularized Skin Construct (Tube-Like Structure Formation)

Another consideration for in vitro fabrication of 3D models is the common problem of loss of cell viability in long-term cultures,⁶² which results from a lack of neovascularization from the peripheral vessels of the wound edge.⁶³ This leads to poor nutrition and oxygen deficiency inside the tissues.⁶⁴ Several studies have confirmed that insufficient vascularization leads to cellular necrosis or complete loosening of implanted skin replacements.^{65, 66} The successful survival of any engineered tissue upon implantation depends on the efficient diffusion of nutrients and oxygen.⁶⁷ Thus, vascularization of engineered tissue is necessary for the fabrication of three-dimensional tissue constructs. The in vivo viability of the construct is maintained by providing nutrients and oxygen and removing waste from living tissues.^{68, 69} Vascularization is a complex process that is based on two fundamental processes, starting with vasculogenesis and terminating in angiogenesis. During vasculogenesis, new blood vessels, or tube-like structures, are formed from the clusters of undifferentiated endothelial cells, after which the angiogenic process initiates the localization of vessel formation by sprouting endothelial cells from pre-existing blood vessels.⁷⁰ This is accomplished through the differentiation of endothelial progenitor cells into mature endothelial cells. During this process, specific proteases are secreted to degrade the extracellular matrix, allowing for the proliferation and polarization of endothelial cells, which ultimately expand toward the vascular tree.⁷¹

Consequently, several strategies have been employed to establish vascularization of the engineered construct. As the growth rate of newly formed microvessels is relatively slow, the angiogenic approach is not suitable for promoting vascularization in massive constructs.⁷²⁻⁷⁴ For that reason, a strategy of in vitro pre-vascularization of tissue constructs before implantation has been established to overcome cellular mortality and enhance the in vivo survival of engineered tissues constructs.⁶⁴ This strategy promotes the anastomosis of pre-vascularized vessels with host vasculature and thus supplies the damaged site with oxygen and nutrients.⁷⁵ In this approach, the construction of capillaries and vessel networks is based on endothelial or endothelial progenitor cells rather than pre-existing blood vessels.⁷⁶ This may result in a more efficient graft and reduces the time needed for the in-growth of angiogenic vascular networks. Also, the longer formation time of in vivo vascularization for unvascularized constructs results in cellular necrosis before the formation of the functional vascular network, and results in premature failure of the construct.⁷⁷ It has been demonstrated that implantation with an unvascularized construct takes longer to become vascularized than implantation of a vascularized construct, as the host vascular system is the only source for the delivery of oxygen and nutrients to the encapsulated cells.⁷⁸ Also, different studies have revealed that pre-vascularized scaffolds facilitate the integration of the scaffold vasculature with the host vasculature.^{79, 80}

Therefore, the pre-vascularization of tissue-engineered constructs before implantation is considered to be vital.⁸¹ The vascularization of skin tissue engineering constructs has been achieved by encapsulating vessel-forming cells within the scaffold.⁸²⁻⁸⁵

Co-culture or multi-culture systems are recent approaches that aim to improve the pre-vascularization of engineered skin tissue by combining vessel-forming cells, such as endothelial cells, with supportive cells, such as fibroblasts or smooth muscle cells. Consequently, blood vessels are formed as the endothelial cells proliferate, migrate and differentiate. In addition, fibroblasts secrete soluble angiogenic growth factors, such as VEGF, platelet drive growth factor (PDGF) and transforming growth factor beta (TGF-β),^{86, 87} which stimulate and promote sprouting of endothelial cells, the formation of lumen and the maintenance of long-term stability of the new vessels.⁸⁸ Inter-cellular lumen formation has been verified within 4-5 days by co-culturing fibroblasts with endothelial cells.88 The differentiation of vascular endothelial cells and the formation of vessel-like structures cannot be achieved with-out appropriate cues.^{89, 90} Therefore, the presence of supportive cells, such as fibroblasts, offers both the extracellular matrix and the angiogenic growth factors necessary for tube-like structure formation.⁹⁰ Moreover, a co-culture system composed of fibroblasts and vascular endothelial cells using type I collagen gel has demonstrated support of endothelial cell differentiation and migration.⁹¹ Also, combining human dermal microvascular endothelial cells, fibroblasts and keratinocytes has been shown to enhance vascularization, promote re-epithelialization and hasten the final remodeling phase of the wound-healing process.⁹² Implantation of pre-vascularized 3D gel with keratinocytes has been shown to accelerate wound healing in mice within 14 days.⁹³ Moreover, a 3D co-culture of fibroblasts and keratinocytes in a plasma gel scaffold has contributed to wound repair. Furthermore, pre-vascularization of this scaffold using endothelial progenitor cells has been shown to accelerate skin wound recovery.⁹³

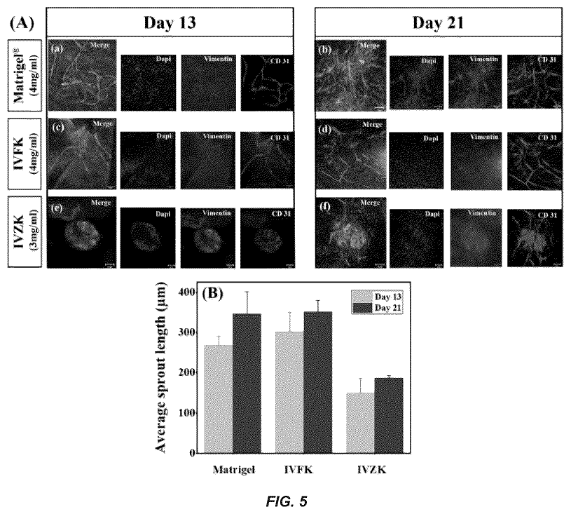

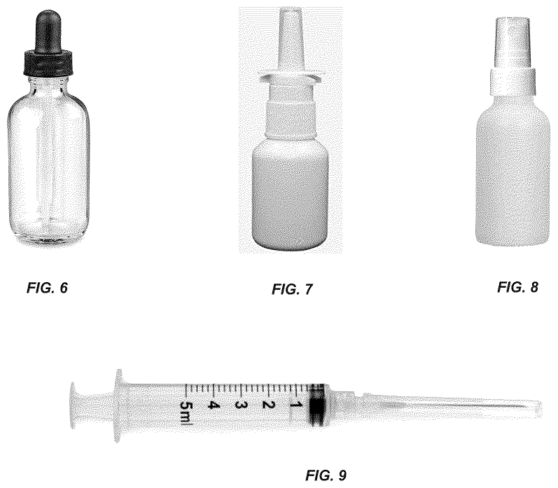

FIG. 5 illustrates the vascularization of co-cultured human skin fibroblasts and HUVECs within different scaffolds. FIG. 5 (A) illustrates cultured in 4 mg mL⁻¹ IVFK nanogel, 3 mg mL⁻¹ IVZK nanogel and 4 mg mL⁻¹ Matrigel, at different time points, respectively. Nucleus stained in blue, fibroblast stained with green fluorescence vimentin and HUVEC with red fluorescence CD31. FIG. 5 (B) illustrates the length of the formed vessel is shown. Scale bars correspond to 100 μm. Complex interconnected capillary-like structures were observed with immunofluorescence staining of co-cultured human dermal fibroblast cells with human umbilical vein cells (HUVEC) on day 13 in IVFK nanogels and Matrigel® (FIG. 5 (A)), while in IVZK nanogels these capillary structures were stagnant in the organization of clusters. Indeed, the assembly of a capillary-like network was promoted and sustained by HDFn at least until day 21 (FIG. 5 (A)). Also, the length of the formed vessels increased throughout this period (FIG. 5 (B)). It is worth mentioning that the length of the tubes in IVFK nanogels was slightly larger than in Matrigel®. Interestingly, the tubes formed in IVFK nanogels started sprouting to form vessel-like structures without any support from vascular growth factors, while IVZK nanogels were still in the vasculogenic stage. This result confirmed that both peptides support vascularization; however, IVZK nanogels may take longer time to reach the angiogenic stage (FIG. 5).

The fabrication of novel 3D skin substitutes by seeding keratinocytes on top of vascularized dermal construct revealed epidermal layer-like structure formations with both peptide hydrogels. It is worth mentioning that basal keratinocytes were mitotically active within all tested materials. However, vascularization in IVZK nanogels was not as good as in Matrigel® and IVFK nanogels, which could be attributed to the peptide sequence modification.

### Peptide Nanogels as Scaffold for Accelerated Wound Healing in Normal Minipigs

A non-healing chronic wound represents a severe complication that can often lead to amputation. As such, there is a clear clinical need for dressings that promote the healing of chronic wounds. An advanced wound dressing aims to keep wound tissues moist while offering increased healing rates, preventing scar formation, reducing pain, minimizing infection, improving cosmetics and lowering overall health care costs. Disclosed embodiments found that peptide nanogels are suitable scaffolds for encapsulating human dermal fibroblasts and keratinocytes. As such, disclosed embodiments used the IVFK and IVZK peptide nanogels as dressings on full-thickness excision wounds. Application of the peptide nanogels on full-thickness minipig wounds demonstrated that the scaffolds are biocompatible and that they did not trigger wound inflammation. This suggests that the scaffolds are safe for topical application. In addition, disclosed embodiments generated in situ silver nanoparticles (AgNPs) within the nanogels to assess their efficacy on minipigs with full-thickness excision wounds. The in-situ generation of the silver nanoparticles from a silver nitrate solution was done solely through UV irradiation, and no reducing agent was used. The monodispersed size of the AgNPs was confirmed by TEM, demonstrating a size range of 10-20 nm, which has been verified as a size that is sufficient to elicit antibacterial activ-ity.^{94, 95} A comparison of the effect of both nanogels, even without the addition of the silver nanoparticles, revealed that the scaffolds themselves have a high potential to act as an antibacterial agent, which may suppress both the inflammatory reaction and activity of proteases. Interestingly, the effect of the peptide nanogels on wound closure was comparable to those of standard care hydrogels. Almost complete re-epithelialization was achieved at day 22, with a gradual reduction in granulation tissue formation.⁹⁵

### Peptide Nanogels as Scaffold for Fabricating 3D Skeletal Muscle Grafts

Skeletal muscle is a soft tissue that constitutes approximately half of human adult body mass.⁹⁶ Muscle mass is profoundly affected by many factors, such as nutritional level, hormonal status, physical activity and illness or injury, which influence the balance of protein synthesis and degradation.⁹⁷ Skeletal muscle is a voluntary moveable tissue that has the ability to convert chemical energy into mechanical energy and then transfer it to tendon tissue. It also supports soft tissue and maintains body posture.⁹⁸ In addition, this tissue carries out various functions of the body, such as respiration, protection of abdominal viscera and control of the movement of limbs.⁹⁹ Skeletal muscle tissue has the native capacity to regenerate/repair through the activation of local satellite cells.^{99, 100} However, this ability declines with age as well as in clinical conditions, such as tumor resection, traumatic sport injuries, including concussions and strains, and muscular dystrophy, which may result in volumetric muscle loss. In these injuries, approximately 20% or more of the muscle mass is lost^{101, 102} and, as a result, tissues lose the ability to signal each other and become unable to repair themselves via natural physiological processes. Thus, surgical intervention is needed¹⁰³⁻¹⁰⁶ to restore normal function and prevent the formation of scar tissue,¹⁰⁴ as the formation of massive scar tissue leads to muscle atrophy and prevents muscle regener-ation.¹⁰⁷ These clinical conditions affect millions of people worldwide and cause significant economic and social problems.^{108, 109} As such, alternative technologies are urgently needed for the reconstruction of skeletal muscle tissues that have experienced volumetric muscle loss and need to regenerate new functional tissue.^{101, 110}

### Biocompatibility of Self-Assembly Ultrashort Peptide Hydrogels on Skeletal Muscle Cells

Disclosed embodiments have used the two synthetically designed novel tetramer peptide biomaterials as scaffolds for this application. Disclosed embodiments applied different biocompatibility assays, including MTT, 3D cell viability and live-dead assays, for cytotoxicity measurement and they all confirmed the biocompatibility of these peptide hydrogels for mouse myoblast cells (C2C12).^{111, 112} Immunofluorescent analysis of cell-laden hydrogels revealed that the proliferation of C2C12 cells was well aligned in the peptide hydrogels compared to the Matrigel® and alginate-gelatin controls. Moreover, myosin heavy chain expression was observed from myoblasts cultured on both scaffolds and was found to be similar to the positive-control Matrigel®. These findings indicate that both scaffolds promote muscle cell differentiation, suggesting that these materials may prove to be beneficial in increasing muscle mass. The fusion index was calculated from myosin heavy chain-stained cells, which was defined as the number of nuclei present in myotubes in comparison to the total number of nuclei present in the observed field. Statistical analysis revealed a significant increase in the number of myotubes containing four or more nuclei in cell-encapsulated IVFK nanogels compared to the other tested materials. In addition, quantitative investigation of cell elongation on the scaffolds was estimated by the cell aspect ratio, which is characterized as the proportion between the length of the longest line and the length of the shortest line across the nuclei. The results demonstrated a slight increase in aspect ratio in 3D culture with hydrogels and control Matrigel® compared to 2D culture. However, this increase did not reach statistical significance.111 In light of these outcomes and the ability to animate myogenesis, it is reasonable to suggest that both hydrogels can be used in the development of techniques to improve promising platforms to enhance skeletal tissue recovery. As such, these materials are suitable for skeletal muscle tissue engineering.

### Peptide Nanogels as Bioinks for Tissue Engineering Applications

Many technologies have been used to generate functional 3D constructs, but none of these methods has succeeded in mimicking the gross native morphology of tissues and organs.¹¹³⁻¹¹⁵ On the contrary, 3D bioprinting is a superior technology due to its accuracy in producing dense, cellularized constructs. It has the additional advantage of producing scalable and customized tissue constructs in a quick and cost-effective manner.¹¹⁶⁻¹¹⁸ On the other hand, the lack of proper biocompatible bioinks with supportive mechanical properties for 3D cell culture is a major obstacle, which results in a lack of cell deposition accuracy and structural organization.117,118 Therefore, new materials with improved physical, mechanical and chemical properties are required to improve tissue engineering applications. Biocompatibility, biodegradability, non-immunogenicity, non-mutagenicity and non-hemolytic scaffolds are essential properties for the long-term culture of cells and engineered tissue for implantation, to avoid adverse physiological side effects. Several biomaterials have been used in vitro as bioinks.^{38, 119} Scaffolds from self-assembling peptides are of particular interest for bioprinting due to their synthetic but natural background. Disclosed embodiments tested the printability of designed biocompatible peptide bioinks by using a commercially available 3D bioprinter. Excellent printability and shape fidelity, two essential standards for 3D bio-printing, were observed with both peptides.^{111, 112} As a proof of concept, circle, square and grid shapes were printed using the peptide bioinks. Inspired by this emerging technology, disclosed embodiments aim to study the printability of the custom-designed robotic 3D bioprinting system^{120, 121} to fabricate 3D scaffolds for the differentiation of myoblast cells. The process of 3D bioprinting is believed to enhance the arrangement of homogeneous cellular scaffolds and improve cell proliferation and adhesion for myotube formation.¹¹² Two sequences of self-assembling peptides are tested and analyzed for cell viability, proliferation and differentiation. The promising results indicate that 3D bioprinting of self-assembling ultrashort peptides may be of value in improving the process of muscle tissue engineering.

### Discussion and Conclusion

Every year, all over the world, hundreds of thousands of patients require hospitalization because of organ failure, which affects quality of life and is costly to treat. Tissue engineering is an alternative approach for creating tis-sue constructs and fabricating functional organs using biological scaffolds. The scaffold is a major component of a tissue-engineered construct, which is made from a biomaterial and has a 3D architecture, in order to accommodate enough cells and promote cell adhesion and proliferation. Recently, fabrication of biomimetic scaffolds has attracted the interest of researchers.¹¹ Among these biomaterials are nanofiber scaffolds, which mimic the natural ECM in terms of its architecture and mechanical properties.²⁷

From this point of view, disclosed embodiments have developed a novel class of rationally designed tetrameric amphiphilic ultrashort peptides, in particular the pep-tides Ac—IVFK—NH₂ (Ac-Ile-Val-Phe-Lys-NH₂) and Ac-IVZK-NH₂ (Ac-Ile-Val-Cha-Lys-NH₂), for the purpose of skin tissue engineering and skeletal muscle tissue engineering. These peptides have an innate tendency to self-assemble into helical nanofibers under aqueous conditions without the need for gelators, cross-linkers or mechanical stimulators. These helical nanofibers organize themselves into supramolecular 3D-meshed nanofibrous networks which resemble the extracellular matrix in terms of its architecture.

The self-assembly of these peptides occurs through antiparallel pairing interactions of two peptide monomers, forming helical intermediate structures. Condensation of these fibers leads to hydrogel scaffold formation.

The gross outcome of the nanofiber network formed from the self-assembly of ultrashort peptide IVFK and IVZK nanogels was confirmed by SEM, with the average diameter being within the range found in the natural ECM (5-300 nm),⁴⁵ which supports their use as scaffolds for skin tissue regeneration.⁴⁶⁻⁴⁹ The mechanical stiffness and stability of both peptide nanogels was determined using oscillatory rheology based on measuring the storage modulus (G′) and loss modulus (G″). The G″ values of IVFK and IVZK nanogels were found to be less than their G′ values indicating the gel state of both samples.⁵⁰

As cellular proliferation, adhesion and the formation of three-dimensional cellular networks are extremely important for tissue repair and regeneration, the cytocompatibility of the peptide nanogels was evaluated using neonatal human dermal fibroblast cells (HDFn), human epidermal keratinocytes (HEKn) and mouse myoblast cells (C2C12). The in vitro investigation demonstrated that exposure of HDFn and C2C12 to different concentrations of peptide nanogels did not affect cell growth when compared to cell growth in tissue culture plates and positive-control Matrigel®. Further, a time-dependent increase in ATP production was observed in 3D-cultured HDFn for 3, 7, 14 and 21 days, as well as in 3D-cultured C2C12 for 2, 6 and 8 days. These results demonstrated that the encapsulated cells were metabolically active. Furthermore, fluorescent staining of the actin cytoskeleton, which serves as direct evidence for the cellular morphology and cytoskeleton structure,¹²² demonstrated that both cell types proliferated and extended within the scaffolds in their first days of encapsulation, and their growth rates rap-idly increased. In addition, a network was created through cell-to-cell junctions, which ultimately resulted in the formation of an extensive network saturating the nanogel matrix. Moreover, the deposition of collagen in histological sections of the dermal fibroblast layer indicated that fibroblasts were not only mitotically active, but also functionally active, by producing their own collagen matrix.

In addition, the above-mentioned peptide hydrogels were used to engineer a skin bilayer through co-culture of fibroblasts and keratinocytes. The construction of the 3D skin model was performed by 3D co-culturing of HDFn with HEKn in both IVFK and IVZK hydrogels. The interaction of these two cell types has been extensively studied.^{123, 124} The outcome of culturing demonstrated that fibroblast cells tend to overgrow when compared to keratinocytes, despite the higher cell density of keratinocytes. In addition, the proliferation of keratinocytes was enhanced when co-cultured with fibroblasts, which might be attributed to the growth factors secreted by co-cultured fibroblast cells that are essential for epidermal cell morphogene-sis,¹²⁵ such as keratinocyte growth factor,^{126, 127} insulin-like growth factor II¹²⁸ and hepatocyte growth factor/scatter factor.¹²⁹ The same observation was demonstrated by others, suggesting that the proliferation of keratinocytes during wound healing could be enhanced by direct contact of keratinocytes with fibroblast constructs.^{123, 124, 130, 131} Additionally, the formation of a skin-like layer was observed when keratinocytes were seeded on fibroblasts for 14 days, which was similar to earlier results obtained by others.¹³1-¹³³ These results confirmed that the growth of keratinocytes does depend on fibroblast or fibroblast secretions.¹³⁴ It is known that cross-talk between the epidermis and dermis layers occurs through paracrine signaling, which maintains homeostasis during tissue repair.¹³⁵ In addition, fibroblasts secrete soluble angiogenic growth factors, such as VEGF, PDGF and TGF-β,^{86, 87} which stimulate and promote the sprouting of endothelial cells, the formation of lumen and the maintenance of the long-term stability of new vessels.⁸⁸ In this study, disclosed embodiments confirmed that direct contact of epidermal keratinocytes with dermal fibroblast construct on 3D scaffolds significantly enhanced epidermal cell proliferation. This enhancement was confirmed by the significant increase in the expression of IL-1α, an initiator of keratinocyte activation, in culture keratinocytes alone, as well as in a 3D co-culture system, which in turn stimulates IL-6, which activates the proliferation of fibroblasts and the production of extracellular matrix components, as well as stimulating the secretion of TGFβ-1 (autocrine signal) and β-FGF (paracrine signal). As a result, TGF-β1 induces the expression of CK14, which is an early differentiation marker of basal keratinocytes that returns the healthy phenotype of the basal keratinocytes in an autocrine manner. In addition, IL-6 and β-FGF, secreted by dermal fibroblast in response to the release of IL-1α, stimulate the proliferation and sprouting of endothelial cells and, consequently, promote angiogenesis/neo-vascularization in a paracrine manner.¹³⁶⁻¹³⁸ These outcomes suggested that enhancement of keratinocyte proliferation was, for the most part, promoted by the cytokines delivered by the two cell types and that both cytokines (IL-1α and TGFβ1) play an important role in fibroblast-stimulated keratinocyte proliferation. Similar observations have been shown by other investigators.¹³⁴ Indeed, the function of fibroblasts in skin tissue is stimulated by the presence of epidermal keratinocytes,¹²⁵ and the growth of keratinocytes cultured with fibroblast occurs via a double paracrine manner.¹³⁹

Most recent studies were performed generating skin substitutes by co-culturing epidermal keratinocytes with dermal fibroblasts within a 3D scaf-fold.¹⁴⁰ However, cell viability was lost in long-term cultures⁶² resulting from poor nutrition and oxygen deficiency inside the construct,⁶⁴ which prevented use of these scaffolds for clinical application. Thus, vascularization of skin substitutes is in high demand. It has been demonstrated that the presence of supportive cells, such as fibroblasts, is required to offer both the extracellular matrix and the angiogenic growth factors necessary for tube-like structure formation.⁹⁰ Improvement of vascularization and enhancement of wound healing has been reported through transplantation of endothelial cells and smooth muscle cells.¹⁴¹ The reconstruction of capillary-like structures within tissue-engineered constructs could solve the main problem of deficiency in vascularization that impedes the development of most tissue-engineered organs. Accordingly, in the present study, pre-vascularization was performed by co-culturing of HDFn with HUVECs using the peptide scaffolds, to deter-mine which of the peptide nanogels initiated endothelial sprout formation to a greater extent. Indeed, the assembly of capillary-like networks might be promoted by HDFn secretion of VEGF,¹⁴² which maintained and sustained this network until day 21 without supplementing the medium with growth factor. Moreover, co-culturing empowers the cells to convey signals by means of paracrine, juxtacrine or gap-junctional signals,¹⁴³ thereby prompting adjusted angiogenic responses.¹⁴⁴⁻¹⁴⁶ Also, a time-dependent increase in the length of the vessels formed was observed. Similar observations have been demonstrated by others after seeding endothelial cells onto confluent fibro-blast lawns.¹⁴⁷ It is worth mentioning that the IVFK scaffold fulfilled the criteria of a tissue-engineered scaffold by maintaining the in vitro proliferation of dermal fibroblasts, enhancing collagen deposition and supporting pre-revascularization of the dermal construct. Therefore, IVFK scaffolds serve as an efficient substrate for vascular tissue engineering. Consequently, this pre-vascularized 3D dermal construct may have great potential for accelerating the wound-healing process, thereby permitting prior anastomosis. Further-more, expanded vessel arrangements in the inside of the embedded structures provide oxygen and nutrients, which are necessary to maintain skin grafts.⁹³

Disclosed embodiments have also successfully fabricated a vascularized skin equivalent by seeding keratinocytes on top of vascularized dermal constructs. The disclosed results showed that both peptide nanogels not only offer a favorable microenvironment for dermal fibroblast, but also provide suitable niches for epidermal keratinocyte growth, as they proliferate and form layer-like skin structures on top of the vascularized constructs after four days.

Based on this observation, disclosed embodiments could confirm that peptide scaffolds provide a favorable microenvironment for adhesion, spreading and proliferation of skin cells, and are thus safe for topical application. As such, disclosed embodiments may evaluate the therapeutic effects of these biomaterials on wound closure.

To study peptide nanogels in vivo, full-thickness incision wounds (1×1 cm) were created on the dorsal back of minipigs by surgical removal of the upper epidermis layer and middle dermis layer. Then the biomaterials and their silver nanoparticle-encapsulated counterparts were applied topically. Disclosed embodiments examined their healing capacity in comparison to commercially available standard-of-care hydrogel dressings, namely DuoDerm® Hydroactive® hydro-gel and ConvaTec's Aquacel® Ag Extra hydrofiber technology. Disclosed embodiments find that application of these hydrogels to chronic full-thickness wounds stimulated granulation tissue formation as well as re-epithelialization, which eventually closed the wound without the addition of exogenous growth factors or cells.^{95, 148} The presence of granulation tissue suggests that both hydrogels support the proliferation of endothelial cells and thus enhance angiogenesis. Also, the observed ability of these peptide nanogels alone, without any additional additive factors, showed a similar effect as the controls. This will enable the future development of 3D scaffolds containing skin/stem cells as well as angiogenic factors to promote the vascularization needed for a successful tissue engineering graft. Moreover, these peptide nanogels, assembled from compounds containing only four amino acids, are less costly due to ease of synthesis and production. They do not need any chemical additives, which is in stark contrast to commercially available hydrogel dressings. Considering the disclosed results, the peptide nanogels utilized are promising materials for the fabrication of skin substitutes as well as 3D skin grafts, particularly in the context of wound healing.

Finally, as a step forward in using these peptides as bioinks for bioprinting applications, disclosed embodiments confirmed the printability of these peptides using an extrusion-based printing method. The results of disclosed embodiments indicate that peptide bioinks are printable and promising candidates for 3D bioprinting of different cell types. These peptide bioinks may create elastically designed and accurately defined structures with a uniform distribution of cells, which could lead to better architectural organization in the development of different tissue engineering applications. In addition, the 3D-bioprinted scaffolds, which simulate the highly complex structures of the ECM, were engineered by a home-developed robotic 3D bioprinter. The cells were infused into the 3D constructs during printing through a custom extrusion method. The two-inlet nozzle, fabricated in-house, allowed gelation of the peptide and even distribution of the cells within each layer of the construct. The results showed that the 3D-printed platforms could enhance cell adhesion and proliferation for at least 5 days, as demonstrated by the live-dead assay. Moreover, they could promote myotube formation and hence induce the myogenic differentiation of C2C12 cells in 3D culture. This confirms the biocompatibility of the 3D-bioprinted structures and suggests that they can potentially be used as cell culture platforms for skeletal tissue engineering and regeneration.

In summary, studies from disclosed embodiments show that newly developed peptide nanogels provide native cues to human dermal fibroblasts as well as mouse myoblast cells and promote proliferation and extensive network formation in vitro. Also, throughout the co-culture, both scaffolds support cellular attachment and provide a substantial increase in epidermal cell proliferation over time, which eventually covers the surface of the entire construct to form skin-like structures. The results represent an improvement in the fabrication of der-mal grafts as well as 3D skin models.

In one embodiment, an ultrashort self-assembling amphiphilic peptide wherein said aliphatic amino acids and said aromatic amino acids and said polar amino acids are either D amino acids or L amino acids, comprising at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

In one embodiment, the peptide is tetrameric amphiphilic self-assembling peptide.

In one embodiment, the peptide has amphiphilic peptide sequences with a hydrophobic tail and a hydrophilic head group.

In one embodiment, the peptide self-assembles into nanofibers that form three-dimensional nanogel.

In one embodiment, the peptide generates macromolecular nanofibrous hydrogel in an aqueous solution.

In one embodiment, the peptide is at least one selected from the group consisting of IVFK, and IVZK.

In one embodiment, the peptide has biocompatibility.

In one embodiment, the peptide has non-cytotoxicity.

In one embodiment, the peptide has non-immunogenicity.

In one embodiment, the peptide assembles into helical nanofibers under aqueous conditions without the need for gelator, cross-linker or mechanical stimulator.

In one embodiment, the peptide having helical nano-fibers organize themselves into supramolecular 3D-meshed nanofibrous networks with an average fiber diameter of around 5-30 nm.

In one embodiment, the fibers structurally resemble collagen fibers.

In one embodiment, the stiffness of the peptide is 10-25 kPa.

In one embodiment, a method of wound treatment comprising applying a material to a subject, wherein the material comprises an ultrashort self-assembling amphiphilic peptide wherein said aliphatic amino acids and said aromatic amino acids and said polar amino acids are either D amino acids or L amino acids, comprising at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

In one embodiment, the subject is a human.

In one embodiment, the subject is selected from the group consisting of a mammal, a reptile, a bird, a fish, an amphibian, and an invertebrate.

In one embodiment, a hydrogel or organogel comprising a peptide, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

In one embodiment, the hydrogel or organogel, which is injectable.

In one embodiment, the hydrogel or organogel is comprised in at least one of a fuel cell, a solar cell, an electronic cell, a biosensing device, a medical device, an implant, a pharmaceutical composition and a cosmetic composition.

In one embodiment, a method of preparing a hydrogel or organogel, the method comprising dissolving a peptide in an aqueous solution or an organic solution, respectively, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

In one embodiment, a kit of parts, the kit comprising a first container with a peptide and a second container with an aqueous or organic solution, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

In one embodiment, a kit comprising an effective amount of a material, wherein the material comprises an ultrashort self-assembling amphiphilic peptide wherein said aliphatic amino acids and said aromatic amino acids and said polar amino acids are either D amino acids or L amino acids, comprising at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group, and wherein the material is applied in at least one selected from the group consisting of 2D-3D printing and 2D-3D molding.

In one embodiment, a wound dressing or wound healing agent comprising a hydrogel or organogel comprising a peptide, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

In one embodiment, a pharmaceutical composition comprising an ultrashort self-assembling amphiphilic peptide wherein said aliphatic amino acids and said aromatic amino acids and said polar amino acids are either D amino acids or L amino acids, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

In one embodiment, a method of wound treatment comprising administering a hydrogel or organogel comprising a peptide to a subject via a device, wherein the peptide comprises at least one peptide selected from a group of peptides having a general formula selected from AₙBₘX, BₘAₙX, XAₙBₘ, and XBₘAₙ, wherein the total number of amino acids of the ultrashort self-assembling amphiphilic peptide wherein said aliphatic amino acids and said aromatic amino acids and said polar amino acids are either D amino acids or L amino acids, does not exceed 7 amino acids; wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

In one embodiment, the device is selected from the group consisting of a container with a dropper/closure device, a squeeze bottle pump spray, an airless and preservative-free spray, an injectable device, and a patch.

In addition, the application of the disclosed ultrashort peptide nanogels on full-thickness wounds in a minipig model demonstrated biocompatibility with the minipig skin tissue, as the peptide nanogels did not trigger wound inflammation. Thus, they can be considered as a safe biomaterial for topical applications. The peptide-based biomaterials exhibited a similar effect on wound closure when compared to the current standard-of-care hydrogels. Moreover, the comparable effect obtained after using hydrogels with and without AgNPs indicated that these materials have a high potential to act as antibacterial agents. The disclosed results suggest that peptide nanogels as wound dressings may significantly enhance chronic wound healing when combined with cells or growth factors that are suitable for skin tissue regeneration. Disclosed embodiments propose that the peptide nanogels may act as potential carriers for HDFn transplantation in in vivo therapies and may be used as promising biomaterials for tissue engineering applications.

Further in vivo studies may be performed to assess how the 3D culture peptide scaffolds work when seeded together with autologous skin cells. Follow-up studies may allow for a more precise evaluation of the fate of the dressings post-grafting. Disclosed embodiments have also shown that both peptides are printable. The ability to print the disclosed peptides and the return of high cell viability within the printed construct will open up the possibility of 3D bioprinting of different cell types in future. The described results may represent an advance in the context of engineering skin tissue and skeletal muscle tissue, providing opportunities to rebuild missing, failing or damaged parts in the future.

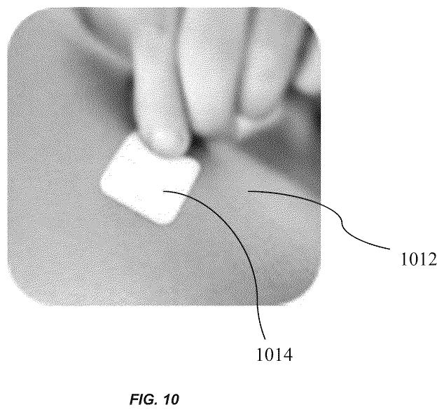

In some examples, a combination disclosed herein may be delivered into a subject's body such as via a patch. FIG. 10 illustrates patch 1014 comprising a combination disclosed herein. Patch 1014 may comprise an adhesive patch for placing on the skin or surfaces 1012 of a subject and one or more active layers embedded in the adhesive patch, wherein the one or more active layers comprise a combination disclosed herein in a therapeutically effective amount. Patch 1014 may be placed on to a subject's body to allow the combination embedded in the adhesive patch be delivered into the subject's bloodstream.

FIG. 10 is an illustrative representation of an exemplary delivery of a disclosed combination into a subject's body through a patch. One of ordinary skill in the art would readily appreciate that any kind of patch suitable for delivering the disclosed products described in the present invention may be utilized.

Having described the many embodiments of the present invention in detail, it will be apparent that modifications and variations are possible without departing from the scope of the invention defined in the appended claims. Furthermore, it should be appreciated that all examples in the present invention, while illustrating many embodiments of the invention, are provided as non-limiting examples and are, therefore, not to be taken as limiting the various aspects so illustrated.

### REFERENCES

The following references are referred to above and are incorporated herein by reference:
1. G. L. Armstrong, L. A. Conn and R. W. Pinner, “Trends in infectious disease mortality in the United States during the 20th century,” JAMA, J Am. Med. Assoc. 281, 61-66 (1999).
2. H. Ben-Bassat, M. Chaouat, N. Segal, E. Zumai, M. Wexler and A. Eldad, “How long can cryopreserved skin be stored to maintain adequate graft performance?,” Burns, 27, 425-431 (2001).
3. S. Aarabi, M. T. Longaker and G. C. Gurtner, “Hypertrophic scar formation following burns and trauma: new approaches to treatment,” PLoS Med., 4, e234 (2007).
4. B. Berman, M. H. Viera, S. Amini, R. Huo and I. S. Jones, “Prevention and management of hypertrophic scars and keloids after burns in children,” J. Craniofac. Surg., 19, 989-1006 (2008).
5. J. Leon-Villapalos, M. Eldardiri and P. Dziewulski, “The use of human deceased donor skin allograft in burn care,” Cell Tissue Banking, 11, 99-104 (2010).
6. R. Lanza, R. Langer and J. Vacanti, “Tissue engineering,” Science, 260, 920-926 (1993).
7. N. T. Elliott and F. Yuan, “A review of three-dimensional in vitro tissue models for drug discovery and transport studies,” J Pharm. Sci., 100, 59-74 (2011).
8. H. Naito, M. Yoshimura, T. Mizuno, S. Takasawa, T. Tojo and S. Taniguchi, “The advantages of three-dimensional culture in a collagen hydrogel for stem cell differentiation,” J. Biomed. Mater. Res., Part A, 101, 2838-2845 (2013).
9. B. M. Baker and C. S. Chen, “Deconstructing the third dimension-how 3D culture microenvironments alter cellular cues,” J. Cell Sci., 125, 3015-3024 (2012).
10. P. A. Soucy and L. H. Romer, “Endothelial cell adhesion, signaling, and morphogenesis in fibroblast-derived matrix,” Matrix Biol., 28, 273-283 (2009).
11. S. G. Kumbar, S. P. Nukavarapu, R. James, L. S. Nair and C. T. Laurencin, “Electrospun poly (lactic acid-co-glycolic acid) scaffolds for skin tissue engineering,” Biomaterials, 29, 4100-4107 (2008).
12. S. Zhou, Y. Wang, K. Zhang, N. Cao, R. Yang and J. Huang, et al., “The Fabrication and Evaluation of a Potential Biomaterial Produced with Stem Cell Sheet Technology for Future Regenerative Medicine,” Stem Cells Int., 2020, 1-12 (2020).
13. A. Rahmani Del Bakhshayesh, N. Annabi, R. Khalilov, A. Akbarzadeh, M. Samiei and E. Alizadeh, et al., “Recent advances on biomedical applications of scaffolds in wound healing and dermal tissue engineering,” Artif Cells, Nanomed., Biotechnol., 46, 691-705 (2018).
14. Y. Garcia, B. Wilkins, R. J. Collighan, M. Griffin and A. Pandit, “Towards development of a dermal rudiment for enhanced wound healing response,” Biomaterials, 29, 857-868 (2008).
15. B. Hafemann, S. Ensslen, C. Erdmann, R. Niedballa, A. Zuhlke and K. Ghofrani, et al., “Use of a collagen/elastin-membrane for the tissue engineering of dermis1,” Burns, 25, 373-384 (1999).
16. P. S. Sahota, J. L. Burn, N. J. Brown and S. MacNeil, “Approaches to improve angiogenesis in tissue-engineered skin,” Wound Repair Regen., 12, 635-642 (2004).
17. U. Kneser, P. M. Kaufmann, H. C. Fiegel, J. M. Pollok, D. Kluth and H. Herbst, et al., “Long-term differentiated function of heterotopically transplanted hepatocytes on three-dimensional polymer matrices,” J Biomed. Mater. Res., The Japanese Society for Biomaterials, and The Australian Society for Biomaterials and the Korean Society for Biomaterials, 47, 494-503 (1999).
18. M. Monsour, R. Mohammed, S. Gorham, D. French and R. Scott, “An assessment of a collagen/vicryl composite membrane to repair defects of the urinary bladder in rabbits,” Urol. Res., 15, 235-238 (1987).
19. T. Hadlock, C. Sundback, D. Hunter, M. Cheney and J. P. Vacanti, “A polymer foam conduit seeded with Schwann cells promotes guided peripheral nerve regeneration,” Tissue Eng., 6, 119-127 (2000).
20. J. F. Hansbrough, J. L. Morgan, G. E. Greenleaf and R. Bartel, “Composite grafts of human keratinocytes grown on a polyglactin mesh-cultured fibroblast dermal substitute function as a bilayer skin replacement in full-thickness wounds on athymic mice,” J. Burn Care Rehabil., 14, 485-494 (1993).
21. S. L. Ishaug-Riley, G. M. Crane-Kruger, M. J. Yaszemski and A. G. Mikos, “Three-dimensional culture of rat calvarial osteoblasts in porous biodegradable polymers,” Biomaterials, 19, 1405-1412 (1998).
22. Y. Cao, J. P. Vacanti, K. T. Paige and J. Upton, “Transplantation of Chondrocytes Utilizing a Polymer-Cell Construct to Produce Tissue-Engineered Cartilage in the shape of a human ear.,” Plast. Reconstr. Surg., 100, 297-302 (1997).
23. V. S. Lin, M. C. Lee, S. O'Neal, J. McKean and K. P. Sung, “Ligament tissue engineering using synthetic biodegradable fiber scaffolds,” Tissue Eng., 5, 443-451 (1999).
24. D. Jiang, J. Liang and P. W. Noble, “Regulation of non-infectious lung injury, inflammation, and repair by the extracellular matrix glycosaminoglycan hyaluronan,” Anat. Rec., 293, 982-985 (2010).
25. C. M. Agrawal and R. B. Ray, “Biodegradable polymeric scaffolds for musculoskeletal tissue engineering,” J Biomed. Mater. Res., The Japanese Society for Biomaterials, and The Australian Society for Biomaterials and the Korean Society for Biomaterials, 55, 141-150 (2001).
26. X. Zong, H. Bien, C.-Y. Chung, L. Yin, D. Fang and B. S. Hsiao, et al., “Electrospun fine-textured scaffolds for heart tissue constructs,” Biomaterials, 26, 5330-5338 (2005).
27. Y. Loo, A. Lakshmanan, M. Ni, L. L. Toh, S. Wang and C. A. Hauser, “Peptide bioink: self-assembling nanofibrous scaffolds for three-dimensional organotypic cultures,” Nano Lett., 15, 6919-6925 (2015).
28. A. Mishra, Y. Loo, R. Deng, Y. J. Chuah, H. T. Hee and J. Y. Ying, et al., “Ultrasmall natural peptides self-assemble to strong temperature-resistant helical fibers in scaffolds suitable for tissue engineering,” Nano Today, 6, 232-239 (2011).
29. C. A. Hauser, R. Deng, A. Mishra, Y. Loo, U. Khoe and F. Zhuang, et al., “Natural tri-to hexapeptides self-assemble in water to amyloid β-type fiber aggregates by unexpected α-helical intermediate structures,” Proc. Natl. Acad. Sci. U.S.A, 108, 1361-1366 (2011).
30. W. Y. Seow and C. A. Hauser, “Short to ultrashort peptide hydrogels for biomedical uses,” Mater. Today, 17, 381-388 (2014).
31. N. Yadav, M. K. Chauhan and V. S. Chauhan, “Short to ultrashort peptide-based hydrogels as a platform for biomedical applications,” Biomater. Sci., 8, 84-100 (2020).
32. Z. Ma, M. Kotaki, R. Inai and S. Ramakrishna, “Potential of nanofiber matrix as tissue-engineering scaffolds,” Tissue Eng., 11, 101-109 (2005).
33. R. Ravichandran, S. Liao, C. C. Ng, C. K. Chan, M. Raghunath and S. Ramakrishna, “Effects of nanotopography on stem cell phenotypes,” World J Stem Cells, 1, 55 (2009).
34. K. Hosoyama, C. Lazurko, M. Muñoz, C. D. McTiernan and E. I. Alarcon, “Peptide-based functional biomaterials for soft-tissue repair,” Front. Bioeng. Biotechnol., 7, 205 (2019).
35. R. Mohammadinejad, A. Kumar, M. Ranjbar-Mohammadi, M. Ashrafizadeh, S. S. Han and G. Khang, et al., “Recent Advances in Natural Gum-Based Biomaterials for Tissue Engineering and Regenerative Medicine: A Review,” Polymers, 12, 176 (2020).
36. T. C. Holmes, “Novel peptide-based biomaterial scaffolds for tissue engineering,” Trends Biotechnol., 20, 16-21 (2002).
37. M. Asadian, K. V. Chan, M. Norouzi, S. Grande, P. Cools and R. Morent, et al., “Fabrication and Plasma Modification of Nanofibrous Tissue Engineering Scaffolds,” Nanomaterials, 10, 119 (2020).
38. C. A. Hauser and S. Zhang, “Designer self-assembling peptide nanofiber biological materials,” Chem. Soc. Rev., 39, 2780-2790 (2010).
39. Y. Loo, S. Zhang and C. A. Hauser, “From short peptides to nanofibers to macromolecular assemblies in biomedicine,” Biotechnol. Adv., 30, 593-603 (2012).
40. E. C. Wu, S. Zhang and C. A. Hauser, “Self-assembling peptides as cell-interactive scaffolds,” Adv. Funct. Mater., 22, 456-468 (2012).
41. C. A. Hauser, R. Deng, A. Mishra, Y. Loo, U. Khoe and F. Zhuang, et al., “Natural tri-to hexapeptides self-assemble in water to amyloid β-type fiber aggregates by unexpected u-helical intermediate structures,” Proc. Natl. Acad. Sci. U.S.A, 1361-1366 (2011).
42. Y. Loo, Y.-C. Wong, E. Z. Cai, C.-H. Ang, A. Raju and A. Lakshmanan, et al., “Ultrashort peptide nanofibrous hydrogels for the acceleration of healing of burn wounds,” Biomaterials, 35, 4805-4814 (2014).
43. L. Bacakova, M. Zikmundova, J. Pajorova, A. Broz, E. Filova, A. Blanquer, et al., “Nanofibrous Scaffolds for Skin Tissue Engineering and Wound Healing Based on Synthetic Polymers. Applications of Nanobiotechnology,” IntechOpen, (2019).
44. M. D. Shoulders and R. T. Raines, “Collagen structure and stability,” Annu. Rev. Biochem., 78, 929-958 (2009).
45. V. Jayawarna, M. Ali, T. A. Jowitt, A. F. Miller, A. Saiani and J. E. Gough, et al., “Nanostructured hydrogels for three-dimensional cell culture through self-assembly of fluorenylmethoxycarbonyl-dipeptides,” Adv. Mater., 18, 611-614 (2006).
46. R. Ellis-Behnke, Y.-X. Liang, S.-W. You, D. Tay, S. Zhang and G. Schneider, et al., “Peptide nanofiber scaffold for brain repair and axon regeneration with functional return of vision. Where do we go from?,” Nanomedicine, 2, 317 (2006).
47. G. C. Gurtner, S. Werner, Y. Barrandon and M. T. Longaker, “Wound repair and regeneration,” Nature, 453, 314-321 (2008).
48. G. Sun, X. Zhang, Y.-I. Shen, R. Sebastian, L. E. Dickinson and K. Fox-Talbot, et al., “Dextran hydrogel scaffolds enhance angiogenic responses and promote complete skin regeneration during burn wound healing,” Proc. Natl. Acad. Sci. U.S.A, 108, 20976-20981 (2011).
49. E. S. Place, N. D. Evans and M. M. Stevens, “Complexity in biomaterials for tissue engineering,” Nat. Mater., 8, 457 (2009).
50. A. Lakshmanan, D. W. Cheong, A. Accardo, E. Di Fabrizio, C. Riekel and C. A. Hauser, “Aliphatic peptides show similar self-assembly to amyloid core sequences, challenging the importance of aromatic interactions in amyloidosis,” Proc. Natl. Acad. Sci. U.S.A, 110, 519-524 (2013).
51. M. A. Greenfield, J. R. Hoffman, M. O. de la Cruz and S. I. Stupp, “Tunable Mechanics of Peptide Nanofiber Gels,” Langmuir, 3641-3647 (2010).
52. T. G. Mezger, “The Rheology Handbook: For Users of Rotational and Oscillatory Rheometers,” Vincentz Network GmbH & Co KG, (2006).
53. S. MacNeil, “Progress and opportunities for tissue-engineered skin,” Nature, 445, 874 (2007).
54. P. M. Elias, “The skin barrier as an innate immune element,” Semin. Immunopathol., 3-14 (2007).
55. G. Menon and A. Kligman, “Barrier functions of human skin: a holistic view,” Skin Pharmacol. Physiol., 22, 178-189 (2009).
56. K. Christensen, G. Doblhammer, R. Rau and J. W. Vaupel, “Ageing populations: the challenges ahead,” Lancet, 374, 1196-1208 (2009).
57. V. Falanga, “Chronic wounds: pathophysiologic and experimental considerations,” J. Invest. Dermatol., 100, 721-725 (1993).
58. F. Zhu, Y. Chen, S. Yang, Q. Wang, F. Liang and X. Qu, et al., “Surface patterned hydrogel film as a flexible scaffold for 2D and 3D cell co-culture,” RSC Adv., 6, 61185-61189 (2016).
59. M. R. Reithofer, A. Lakshmanan, A. T. Ping, J. M. Chin and C. A. Hauser, “In situ synthesis of size-controlled, stable silver nanoparticles within ultrashort peptide hydrogels and their anti-bacterial properties,” Biomaterials, 35, 7535-7542 (2014).
60. W. T. Arab, H. H. Susapto and C. A. Hauser, “Peptide Nanogels as Scaffold for Fabricating Dermal Graft and 3D Skin Models,” Manuscript under submission, (2020).
61. J.-L. Ruan, N. L. Tulloch, V. Muskheli, E. E. Genova, P. D. Mariner and K. S. Anseth,
et al., “An improved cryosection method for polyethylene glycol hydrogels used in tissue engineering,” Tissue Eng., Part C, 19, 794-801 (2013).
62. A. S. Burke, L. A. MacMillan-Crow and J. A. Hinson, “The hepatocyte suspension assay is superior to the cultured hepatocyte assay for determining mechanisms of acetaminophen hepatotoxicity relevant to in vivo toxicity,” Chem. Res. Toxicol., 23, 1855-1858 (2010).
63. S. Xiong, X. Zhang, P. Lu, Y. Wu, Q. Wang and H. Sun, et al., “A gelatin-sulfonated silk composite scaffold based on 3D printing technology enhances skin regeneration by stimulating epidermal growth and dermal neovascularization,” Sci. Rep., 7, 4288 (2017).
64. J. Samal, S. Weinandy, A. Weinandy, M. Helmedag, L. Rongen and B. Hermanns-Sachweh, et al., “Co-Culture of Human Endothelial Cells and Foreskin Fibroblasts on 3D Silk-Fibrin Scaffolds Supports Vascularization,” Macromol. Biosci., 15, 1433-1446 (2015).
65. P. S. Sahota, J. L. Burn, M. Heaton, E. Freedlander, S. K. Suvarna and N. J. Brown, et al., “Development of a reconstructed human skin model for angiogenesis,” Wound Repair Regen., 11, 275-284 (2003).
66. B. Hendrickx, J. J. Vranckx and A. Luttun, “Cell-based vascularization strategies for skin tissue engineering,” Tissue Eng., Part B, 17, 13-24 (2010).
67. P. Carmeliet and R. K. Jain, “Angiogenesis in cancer and other diseases,” Nature, 407, 249 (2000).
68. C. O. Cassell, O. S. Hofer, W. A. Morrison and K. R. Knight, “Vascularisation of tissue-engineered grafts: the regulation of angiogenesis in reconstructive surgery and in disease states, Br. J. Plast. Surg., 55, 603-610 (2002).
69. T. Kaully, K. Kaufman-Francis, A. Lesman and S. Levenberg, “Vascularization—the conduit to viable engineered tissues,” Tissue Eng., Part B, 15, 159-169 (2009).
70. W. Risau, “Mechanisms of angiogenesis,” Nature, 386, 671 (1997).
71. W. Risau and I. Flamme, “Vasculogenesis,” Annu. Rev. Cell Dev. Biol., 11, 73-91 (1995).
72. M. W. Laschke and M. D. Menger, “Prevascularization in tissue engineering:current concepts and future directions,” Biotechnol. Adv., 34, 112-121 (2016).
73. M. W. Laschke, Y. Harder, M. Amon, I. Martin, J. Farhadi and A. Ring, et al., “Angiogenesis in tissue engineering: breathing life into constructed tissue substitutes,” Tissue Eng., 12, 2093-2104 (2006).
74. U. Utzinger, B. Baggett, J. A. Weiss, J. B. Hoying and L. T. Edgar, “Large-scale time series microscopy of neovessel growth during angiogenesis,” Angiogenesis, 18, 219-232 (2015).
75. G. M. Mitchell and W. A. Morrison, “In Vivo Vascularization for Large-Volume Soft Tissue Engineering,” Vascularization: Regenerative Medicine and Tissue Engineering, ed. E. M Brey, CRC Press, 343 (2014).
76. J. Baldwin, M. Antille, U. Bonda, E. M. De-Juan-Pardo, K. Khosrotehrani and S. Ivanovski, et al., “In vitro pre-vascularisation of tissue-engineered constructs A co-culture perspective,” Vasc. Cell, 6, 13 (2014).
77. A. W. L. Liew and Y. Zhang, “In vitro pre-vascularization strategies for tissue engineered constructs-Bioprinting and others,” Int. J Bioprint., 3, 3-17 (2017).
78. W. Zhang, L. S. Wray, J. Rnjak-Kovacina, L. Xu, D. Zou and S. Wang, et al., “Vascularization of hollow channel-modified porous silk scaffolds with endothelial cells for tissue regeneration,” Biomaterials, 56, 68-77 (2015).
79. J. L. West and J. J. Moon, “Vascularization of engineered tissues: approaches to promote angiogenesis in biomaterials,” Curr. Top. Med. Chem., 8, 300-310 (2008).
80. J. D. Baranski, R. R. Chaturvedi, K. R. Stevens, J. Eyckmans, B. Carvalho and R. D. Solorzano, et al., “Geometric control of vascular networks to enhance engineered tissue integration and function,” Proc. Natl. Acad. Sci. U.S.A, 110, 7586-7591 (2013).
81. J. Rouwkema, N. C. Rivron and C. A. van Blitterswijk, “Vascularization in tissue engineering,” Trends Biotechnol., 26, 434-441 (2008).
82. A. F. Black, F. Berthod, N. L'heureux, L. Germain and F. A. Auger, “In vitro reconstruction of a human capillary-like network in a tissue-engineered skin equivalent,” FASEB J, 12, 1331-1340 (1998).
83. D. M. Supp, K. Wilson-Landy and S. T. Boyce, “Human dermal microvascular endothelial cells form vascular analogs in cultured skin substitutes after grafting to athymic mice,” FASEB J., 16, 797-804 (2002).
84. P. L. Tremblay, V. Hudon, F. Berthod, L. Germain and F. A. Auger, “Inosculation of tissue-engineered capillaries with the host's vasculature in a reconstructed skin transplanted on mice,” Am. J Transplant., 5, 1002-1010 (2005).
85. H. K. Wong, C. R. I. Lam, F. Wen, S. K. M. Chong, N. S. Tan and C. Jerry, et al., “Novel method to improve vascularization of tissue engineered constructs with biodegradable fibers,” Biofabrication, 8, 015004 (2016).
86. S. Kellouche, S. Mourah, A. Bonnefoy, D. Schoëvaert, M.-P. Podgomiak and F. Calvo, et al., “Platelets, thrombospondin-1 and human dermal fibroblasts cooperate for stimulation of endothelial cell tubulogenesis through VEGF and PAI-1 regulation,” Exp. Cell Res., 313, 486-499 (2007).
87. V. Paunescu, F. M. Bojin, C. A. Tatu, O. I. Gavriliuc, A. Rosca and A. T. Gruia, et al., “Tumour-associated fibroblasts and mesenchymal stem cells: more similarities than differences,” J. Cell. Mol. Med., 15, 635-646 (2011).
88. A. C. Newman, M. N. Nakatsu, W. Chou, P. D. Gershon and C. C. Hughes, “The requirement for fibroblasts in angiogenesis: fibroblast-derived matrix proteins are essential for endothelial cell lumen formation,” Mol. Biol. Cell, 22, 3791-3800 (2011).
89. R. Auerbach, R. Lewis, B. Shinners, L. Kubai and N. Akhtar, “Angiogenesis assays: a critical overview,” Clin. Chem., 49, 32-40 (2003).
90. C. A. Staton, S. M. Stribbling, S. Tazzyman, R. Hughes, N. J. Brown and C. E. Lewis, “Current methods for assaying angiogenesis in vitro and in vivo,” Int. J. Exp. Pathol., 85, 233-248 (2004).
91. R. Montesano, M. S. Pepper and L. Orci, Paracrine induction of angiogenesis in vitro by Swiss 3T3 fibroblasts, J. Cell Sci., 105, 1013-1024 (1993).
92. M. Cerqueira, R. P. Pirraco, A. Martins, T. Santos, R. Reis and A. Marques, “Cell sheet technology-driven re-epithelialization and neovascularization of skin wounds,” Acta Biomater., 10, 3145-3155 (2014).
93. N.-T. Dai, W.-S. Huang, F.-W. Chang, L.-G. Wei, T.-C. Huang and J.-K. Li, et al., “Development of a Novel Pre-Vascularized Three-Dimensional Skin Substitute Using Blood Plasma Gel,” Cell Transplant., 27, 1535-1547 (2018).
94. F. Martinez-Gutierrez, P. L. Olive, A. Banuelos, E. Orrantia, N. Nino and E. M. Sanchez, et al., “Synthesis, characterization, and evaluation of antimicrobial and cytotoxic effect of silver and titanium nanoparticles,” Nanomedicine, 6, 681-688 (2010).
95. W. T. Arab, A. M. Niyas, K. Seferji, H. H. Susapto, I. Alexander and I. Cima, et al., “Evaluation of Peptide Nanogels for Accelerated Wound Healing in Normal Micropigs,” Front. Nanosci. Nanotechnol., 4, 1-9 (2018).
96. B. J. Kwee and D. J. Mooney, “Biomaterials for skeletal muscle tissue engineering,” Curr. Opin. Biotechnol., 47, 16-22 (2017).
97. W. R. Frontera and J. Ochala, “Skeletal muscle: a brief review of structure and function,” Calcif Tissue Int., 96, 183-195 (2015).
98. M. Beldjilali-Labro, A. Garcia Garcia, F. Farhat, F. Bedoui, J.-F. Grosset and M. Dufresne, et al., “Biomaterials in Tendon and Skeletal Muscle Tissue Engineering: Current Trends and Challenges,” Materials, 11, 1116 (2018).
99. F. Relaix and P. S. Zammit, “Satellite cells are essential for skeletal muscle regeneration: the cell on the edge returns centre stage,” Development, 139, 2845-2856 (2012).
100. A. S. Brack and T. A. Rando, “Tissue-specific stem cells: lessons from the skeletal muscle satellite cell,” Cell Stem Cell, 10, 504-514 (2012).
101. B. F. Grogan, J. R. Hsu and S. T. R. “Consortium, Volumetric muscle loss,” J. Am. Acad. Orthop. Surg., 19, S35-S37 (2011).
102. N. J. Turner and S. F. Badylak, “Regeneration of skeletal muscle,” Cell Tissue Res., 347, 759-774 (2012).
103. G. S. Lynch, J. D. Schertzer and J. G. Ryall, “Anabolic agents for improving muscle regeneration and function after injury,” Clin. Exp. Pharmacol. Physiol., 35, 852-858 (2008).
104. T. A. Jarvinen, T. L. Jarvinen, M. Kaariainen, V. Aarimaa, S. Vaittinen and H. Kalimo, et al., “Muscle injuries: optimising recovery,” Best Pract. Res., Clin. Rheumatol., 21, 317-331 (2007).
105. T. A. Jarvinen, T. L. Jarvinen, M. Kaariainen, H. Kalimo and M. Jarvinen, “Muscle injuries: biology and treatment,” Am. J. Sports Med., 33, 745-764 (2005).
106. T. A. Jarvinen, M. Kaariainen, M. Jarvinen and H. Kalimo, “Muscle strain injuries,” Curr. Opin. Rheumatol., 12, 155-161 (2000).
107. B. T. Corona, X. Wu, C. L. Ward, J. S. McDaniel, C. R. Rathbone and T. J. Walters, “The promotion of a functional fibrosis in skeletal muscle with volumetric muscle loss injury following the transplantation of muscle-ECM,” Biomaterials, 34, 3324-3335 (2013).
108. H. Manring, E. Abreu, L. Brotto, N. Weisleder and M. Brotto, “Novel excitation-contraction coupling related genes reveal aspects of muscle weakness beyond atrophy—new hopes for treatment of musculoskeletal diseases,” Front. Physiol., 5, 37 (2014).
109. J. M. Grasman, M. J. Zayas, R. L. Page and G. D. Pins, “Biomimetic scaffolds for regeneration of volumetric muscle loss in skeletal muscle injuries,” Acta Biomater., 25, 2-15 (2015).
110. V. J. Mase, J. R. Hsu, S. E. Wolf, J. C. Wenke, D. G. Baer and J. Owens, et al., “Clinical application of an acellular biologic scaffold for surgical repair of a large, traumatic quadriceps femoris muscle defect,” Orthopedics, 33, 511 (2010).
111. W. Arab, S. Rauf, O. Al-Harbi and C. Hauser, “Novel Ultrashort Self-Assembling Peptide Bioinks for 3D Culture of Muscle Myoblast Cells,” Int. J. Bioprint., 4, 129 (2018).
112. W. Arab, K. Kahin, Z. Khan and C. Hauser, “Exploring nanofibrous self-assembling peptide hydrogels using mouse myoblast cells for three-dimensional bioprinting and tissue engineering applications,” Int. J. Bioprint., 198 (2019).
113. I. Shadrin, A. Khodabukus and N. Bursac, “Striated muscle function, regeneration, and repair,” Cell. Mol. Life Sci., 73, 4175-4202 (2016).
114. C. Fuoco, L. L. Petrilli, S. Cannata and C. Gargioli, “Matrix scaffolding for stem cell guidance toward skeletal muscle tissue engineering,” J Orthop. Surg. Res., 11, 86 (2016).
115. C. Fuoco, S. Cannata and C. Gargioli, “Could a functional artificial skeletal muscle be useful in muscle wasting?,” Curr. Opin. Clin. Nutr. Metab. Care, 19, 182-187 (2016).
116. V. Mironov, V. Kasyanov, C. Drake and R. R. Markwald, “Organ Printing: Promises and Challenges,” Regener. Med., 93-103 (2008).
117. H.-W. Kang, S. J. Lee, I. K. Ko, C. Kengla, J. J. Yoo and A. Atala, “A 3D bioprinting system to produce human-scale tissue constructs with structural integrity,” Nat. Biotechnol., 34, 312 (2016).
118. S. V. Murphy and A. Atala, “3D bioprinting of tissues and organs,” Nat. Biotechnol., 32, 773 (2014).
119. D. Sundaramurthi, S. Rauf and C. Hauser, “3D bioprinting technology for regenerative medicine applications,” Int. J. Bioprint., 2, 9-26 (2016).
120. K. Kahin, Z. Khan, M. Albagami, S. Usman, S. Bahnshal, H. Alwazani, et al.,
“Development of a robotic 3D bioprinting and microfluidic pumping system for tissue and organ engineering,” Proceedings of SPIE Microfluidics, BioMEMS, and Medical Microsystems XVII, vol. 12, 10875Q1-10875Q10 (2019).
121. Z. Khan, K. Kahin, S. Rauf, G. Ramirez Calderon, N. Papagiannis and M. Abdulmajid, et al., “Optimization of a 3D bioprinting process using ultrashort peptide bioinks,” Int. J. Bioprint., 5, 173 (2018).
122. R. Dominguez and K. C. Holmes, “Actin Structure and Function,” Annu. Rev. Biophys., 169-186 (2011).
123. G. Sawicki, Y. Marcoux, K. Sarkhosh, E. E. Tredget and A. Ghahary, “Interaction of keratinocytes and fibroblasts modulates the expression of matrix metalloproteinases-2 and-9 and their inhibitors,” Mol. Cell. Biochem., 269, 209-216 (2005).
124. J. Sugai, Y. Katakai, M. Iizuka, Y. Kawakubo, A. Ozawa and M. Ohkido, et al., “Interaction of cultured keratinocytes and fibroblasts from human psoriatic and nor mal skin in immunodeficient mice,” J. Dermatol. Sci., 17, 190-197 (1998).
125. R. P. Witte and W. J. Kao, “Keratinocyte-fibroblast paracrine interaction: the effects of substrate and culture condition,” Biomaterials, 26, 3673-3682 (2005).
126. J. S. Rubin, H. Osada, P. W. Finch, W. G. Taylor, S. Rudikoff and S. A. Aaronson, “Purification and characterization of a newly identified growth factor specific for epithelial cells,” Proc. Natl. Acad. Sci. U.S.A, 86, 802-806 (1989).
127. P. W. Finch, J. S. Rubin, T. Miki, D. Ron and S. A. Aaronson, “Human KGF is FGF-related with properties of a paracrine effector of epithelial cell growth,” Science, 245(4919), 752-755 (1989).
128. A. Barreca, M. De Luca, P. D. Monte, S. Bondanza, G. Damonte and G. Cariola, et al., “In vitro paracrine regulation of human keratinocyte growth by fibroblast-derived insulin-like growth factors,” J Cell. Physiol., 151, 262-268 (1992).
129. K. Matsumoto, K. Hashimoto, K. Yoshikawa and T. Nakamura, “Marked stimulation of growth and motility of human keratinocytes by hepatocyte growth factor,” Exp. Cell Res., 196(1), 114-120 (1991).
130. Z. Wang, Y. Wang, F. Farhangfar, M. Zimmer and Y. Zhang, “Enhanced keratinocyte proliferation and migration in co-culture with fibroblasts,” PLoS One, 7, e40951 (2012).
131. S. Huang, Y. Xu, C. Wu, D. Sha and X. Fu, “In vitro constitution and in vivo implantation of engineered skin constructs with sweat glands,” Biomaterials, 31, 5520-5525 (2010).
132. T. Uchino, T. Takezawa and Y. Ikarashi, “Reconstruction of three-dimensional human skin model composed of dendritic cells, keratinocytes and fibroblasts utilizing a handy scaffold of collagen vitrigel membrane,” Toxicol. In Vitro, 23, 333-337 (2009).
133. F. Laco, M. Kun, H. J. Weber, S. Ramakrishna and C. K. Chan, “The dose effect of human bone marrow-derived mesenchymal stem cells on epidermal development in organotypic co-culture,” J. Dermatol. Sci., 55, 150-160 (2009).
134. Z. Wang, Y. Wang, F. Farhangfar, M. Zimmer and Y. Zhang, “Enhanced keratinocyte proliferation and migration in co-culture with fibroblasts,” PLoS One, 7, e40951 (2012).
135. D. Breitkreutz, N. Mirancea and R. Nischt, “Basement membranes in skin: unique matrix structures with diverse functions?,” Histochem. Cell Biol., 132, 1-10 (2009).
136. K. R. Swartz, F. Liu, D. Sewell, T. Schochet, I. Campbell and M. Sandor, et al., “Interleukin-6 promotes post-traumatic healing in the central nervous system,” Brain Res., 896, 86-95 (2001).
137. D. Fee, D. Grzybicki, M. Dobbs, S. Ihyer, J. Clotfelter and S. Macvilay, et al., “Interleukin 6 promotes vasculogenesis of murine brain microvessel endothelial cells,” Cytokine, 12, 655-665 (2000).
138. M. Oberringer, C. Meins, M. Bubel and T. Pohlemann, “A new in vitro wound model based on the co-culture of human dermal microvascular endothelial cells and human dermal fibroblasts,” Biol. Cell, 99, 197-207 (2007).
139. N. Maas-S zabowski, H.-J. Stark and N. E. Fusenig, “Keratinocyte growth regulation in defined organotypic cultures through IL-1-induced keratinocyte growth factor expression in resting fibroblasts,” J Invest. Dermatol., 114, 1075-1084 (2000).
140. Y. Liu, H. Luo, X. Wang, A. Takemura, Y. R. Fang and Y. Jin, et al., “In vitro construction of scaffold-free bilayered tissue-engineered skin containing capillary networks,” BioMed Res. Int., 2013, 1-8 (2013).
141. K. L. Kim, S.-H. Song, K.-S. Choi and W. Suh, “Cooperation of endothelial and smooth muscle cells derived from human induced pluripotent stem cells enhances neovascularization in dermal wounds,” Tissue Eng., Part A, 19, 2478-2485 (2013).
142. P. Brenneisen, R. Blaudschun, J. Gille, L. Schneider, R. Hinrichs and M. Wlaschek, et al., “Essential role of an activator protein-2 (AP-2)/specificity protein 1 (Spl) cluster in the UVB-mediated induction of the human vascular endothelial growth factor in HaCaT keratinocytes,” Biochem. J, 369, 341-349 (2003).
143. J. Hendriks, J. Riesle and C. A. van Blitterswijk, “Co-culture in cartilage tissue engineering,” J. Tissue Eng. Regener. Med., 1, 170-178 (2007).
144. A. Wenger, N. Kowalewski, A. Stahl, A. T. Mehlhorn, H. Schmal and G. B. Stark, et al., “Development and characterization of a spheroidal coculture model of endothelial cells and fibroblasts for improving angiogenesis in tissue engineering,” Cells Tissues Organs, 181, 80-88 (2005).
145. A. Wenger, A. Stahl, H. Weber, G. Finkenzeller, H. Augustin and G. Stark, et al., “Modulation of in vitro angiogenesis in a three-dimensional spheroidal coculture model for bone tissue engineering,” Tissue Eng., 10, 1536-1547 (2004).
146. T. Korff, S. Kimmina, G. Martiny-baron and H. G. Augustin, “Blood vessel maturation in a 3-dimensional spheroidal coculture model: direct contact with smooth muscle cells regulates endothelial cell quiescence and abrogates VEGF responsiveness,” FASEB J., 15, 447-457 (2001).
147. J. M. Sorrell, M. A. Baber and A. I. Caplan, “A self-assembled fibroblast-endothelial cell co-culture system that supports in vitro vasculogenesis by both human umbilical vein endothelial cells and human dermal microvascular endothelial cells,” Cells Tissues Organs, 186, 157-168 (2007).
148. N. C. Carrejo, A. N. Moore, T. L. Lopez Silva, D. G. Leach, I.-C. Li and D. R. Walker, et al., “Multidomain Peptide Hydrogel Accelerates Healing of Full-Thickness Wounds in Diabetic Mice,” ACS Biomater. Sci. Eng., 4, 1386-1396 (2018).

All documents, patents, journal articles and other materials cited in the present application are incorporated herein by reference.

While the present invention has been disclosed with references to certain embodiments, numerous modifications, alterations, and changes to the described embodiments are possible without departing from the sphere and scope of the present invention, as defined in the appended claims. Accordingly, it is intended that the present invention is not limited to the described embodiments, but that it has the full scope defined by the language of the following claims, and equivalents thereof.

1. An ultrashort self-assembling amphiphilic peptide wherein said aliphatic amino acids and said aromatic amino acids and said polar amino acids are either D amino acids or L amino acids, comprising:
at least one peptide selected from a group of peptides having a general formula selected from AₙBₘX, BₘAₙX, XAₙBₘ, and XBₘAₙ,
wherein the total number of amino acids of the ultrashort self-assembling amphiphilic peptide wherein said aliphatic amino acids and said aromatic amino acids and said polar amino acids are either D amino acids or L amino acids, does not exceed 7 amino acids;
wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and
wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

2. The ultrashort self-assembling amphiphilic peptide of claim 1, wherein the peptide is a tetrameric amphiphilic self-assembling peptide.

3. The ultrashort self-assembling amphiphilic peptide of claim 1, wherein the peptide has amphiphilic peptide sequences with a hydrophobic tail and a hydrophilic head group.

4. The ultrashort self-assembling amphiphilic peptide of claim 1, wherein the peptide self-assembles into nanofibers that form three-dimensional nanogel.

5. The ultrashort self-assembling amphiphilic peptide of claim 1, wherein the peptide generates macromolecular nanofibrous hydrogel in an aqueous solution.

6. The ultrashort self-assembling peptide of claim 1, wherein the peptide is at least one selected from the group consisting of IVFK, and IVZK.

7. (canceled)

8. (canceled)

9. (canceled)

10. (canceled)

11. The ultrashort self-assembling amphiphilic peptide of claim 1, wherein the peptide having helical nano-fibers organize themselves into supramolecular 3D-meshed nanofibrous networks with an average fiber diameter of around 5-30 nm.

12. (canceled)

13. (canceled)

14. A method of wound treatment comprising:
applying a material to a subject,
wherein the material comprises an ultrashort self-assembling amphiphilic peptide wherein said aliphatic amino acids and said aromatic amino acids and said polar amino acids are either D amino acids or L amino acids, comprising:
at least one peptide selected from a group of peptides having a general formula selected from AₙBₘX, BₘAₙX, XAₙBₘ, and XBₘAₙ,
wherein the total number of amino acids of the ultrashort self-assembling amphiphilic peptide wherein said aliphatic amino acids and said aromatic amino acids and said polar amino acids are either D amino acids or L amino acids, does not exceed 7 amino acids;
wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and
wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

15. The method of claim 14, wherein the subject is a human.

16. The method of claim 14, wherein the subject is selected from the group consisting of a mammal, a reptile, a bird, a fish, an amphibian, and an invertebrate.

17. A hydrogel or organogel comprising the peptide of claim 1.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A method of preparing a hydrogel or organogel comprising dissolving a peptide in an aqueous solution or an organic solution, respectively, wherein the peptide comprises: at least one peptide selected from a group of peptides having a formula selected from AₙBₘX, BₘAₙX, XAₙBₘ, and XBₘAₙ,
wherein the total number of amino acids of the ultrashort self-assembling does amphiphilic peptide wherein said aliphatic amino acids and said aromatic amino acids and said polar amino acids are either D amino acids or L amino acids, not exceed 7 amino acids;
wherein A is an aliphatic amino acids, selected from the group consisting of: isoleucine, leucine, norleucine, cyclohexylalanine, valine, alanine, glycin, homoallylglycine and homopropargylglycine or any combination thereof, with n being an integer being selected from 0-5; wherein B is comprised of at least one aromatic amino acid selected from the group consisting of: tyrosine, tryptophan, phenylalanine, L-DOPA, or comprised of a peptidomimetic amino acid that is the aliphatic counterpart of the aromatic amino acid, which is the counterpart of amino acid phenylalanine with m being an integer being selected from 0-3; and
wherein X is comprised of a polar amino acid, selected from the group consisting of: aspartic acid, glutamic acid, lysine, arginine, histidine, cysteine, serine, threonine, asparagine, and glutamine, 5-N-ethyl glutamine (theanine), citrulline, thio citrulline, homocysteine, methionine, ethionine, selenomethionine, telluromethionine, allo threonine, homoserine, homoarginine, ornithine, N(6) carboxymethyllysine, or any combination thereof, where the polar group is selected from a hydroxyl, an ether, a carboxyl, an imido, an amido, an amino, an ester, a guanidino, a thio, a thioether, a seleno, and a telluro group.

32. The method of claim 31, wherein the peptide is a tetrameric amphiphilic self-assembling peptide.

33. The method of claim 31, wherein the peptide has amphiphilic peptide sequences with a hydrophobic tail and a hydrophilic head group.

34. (canceled)

35. (canceled)

36. The method of claim 31, wherein the peptide is at least one selected from the group consisting of IVFK, and IVZK.

37. (canceled)

38. (canceled)

39. (canceled)

40. (canceled)

41. (canceled)

42. (canceled)

43. A kit of parts, wherein the kit comprises a first container with the peptide of claim 1 and a second container with an aqueous or organic solution.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A kit comprising an effective amount of a material, wherein the material comprises the peptide of claim 1 and
wherein the material is applied in at least one selected from the group consisting of 2D-3D printing and 2D-3D molding.

56. A wound dressing or wound healing agent comprising a hydrogel or organogel comprising the peptide of claim 1.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. A pharmaceutical composition comprising the peptide of claim 1.

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. A method of wound treatment comprising:
administering a hydrogel or organogel comprising the peptide of claim 1 to a subject via a device.

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)
